(19) United States
(12) Patent Application Publication
(10) Pub. No.: US 2009/0291988 A1
(43) Pub. Date: Nov. 26, 2009

(54) Reversible Inhibitors of Monoamine Oxidase A and B

(76) Inventor: Renata Oballa, Kirkland (CA)

Correspondence Address: MERCK AND CO., INC, P O BOX 2000, RAHWAY, NJ, 07065-0907, US

(21) Appl. No.: 11/922,120

(22) PCT Filed: Jun. 14, 2006
(86) PCT No.: PCT/CA06/00981

§ 371 Date: Dec. 13, 2007

Related U.S. Application Data
(60) Provisional application No. 60/690,411, filed on Jun. 14, 2005.

(51) Int. Cl. A61K 31/165; C07C 255/46; C07C 235/34; C07C 237/24; C07D 213/56; C07C 211/27; A61K 31/275; A61K 31/4418; A61K 31/137; A61P 25/00

(52) U.S. Cl. 514/357; 558/410; 564/171; 564/164; 546/337; 564/442; 514/646; 514/622; 514/620

(57) ABSTRACT

The instant invention relates to compounds of formula I, diagrammed below, wherein R3, E, D and Y are defined in the application, which are useful as reversible inhibitors of monoamine oxidase-B and/or monoamine oxidase-A, and therefore useful to treat or prevent neurological diseases or conditions in mammals, preferably humans.

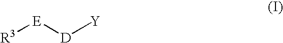

### BACKGROUND OF THE INVENTION

The catecholamine-oxidizing enzyme monoamine oxidase-B (MAO-B) has been hypothesized to be an important determining factor in neurological disorders such as Parkinson's disease. MAO-B regulates levels of brain neurotransmitters, including dopamine. Catalysis of neurotransmitters by monamine oxidase also produces hydrogen peroxide which is a primary originator of oxidative stress which in turn can lead to cellular damage. Inhibition of MAO-B, along with supplementation of dopamine via levodopa, is one of the major antiparkinsonian therapies currently in use. Current MAO-B inhibitors (propargylamines) are irreversible an have also been shown to bind to GAPDH.

Inhibitors of monoamine oxidase-A (MAO-A) are useful for the treatment of depression and anxiety as MAO-A predominantly metabolizes neurotransmitters considered to be important in these disorders. MAO-A inhibitors may also be useful for the treatment of panic disorder, obsessive-compulsive disorder and post-traumatic stress disorder. Reversible monoamine oxidase A inhibitors such as moclobamide are useful for the treatment of depression and anxiety and have a lower propensity to cause hypertension than irreversible MAO-A inhibitors.

### SUMMARY OF THE INVENTION

The instant invention relates to compounds which are useful as reversible inhibitors of MAO-B and/or MAO-A. One embodiment of the present invention is illustrated by a compound of Formula I, and the pharmaceutically acceptable salts, esters, stereoisomers and N-oxide derivatives thereof:

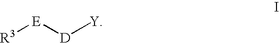

### DETAILED DESCRIPTION OF THE INVENTION

The present invention relates to compounds of the following formula:

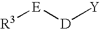

wherein Y is hydrogen, C(R¹)(R²)X, C(O)R¹, C(O)R², C(O)OR¹, CH(OH)R², (C₁₋₆alkyl)C(O)CR¹R²OH, (C₁₋₆alkyl)CR¹R²OH, (C₁₋₆alkyl)OH, SO₂R², C₁₋₆ alkyl, aryl, heteroaryl, C₃₋₈ cycloalkyl or heterocyclyl wherein each said aryl, heteroaryl, cycloalkyl and heterocyclyl groups, which may be monocyclic or bicyclic, is optionally substituted on either the carbon or the heteroatom with one to five substituents independently selected from C₁₋₆ alkyl, halo, cyano or hydroxyl;
X is hydrogen, NH₂ or OH;
R¹ is hydrogen or C₁₋₆ alkyl which is optionally substituted with one to six halo, hydroxyl, O(C₁₋₆ alkyl) or carbonyl;
R² is hydrogen, C₁₋₆ alkyl, aryl, heteroaryl, arylalkyl, heteroarylalkyl or hydroxyl wherein said alkyl, aryl, heteroaryl, haloalkyl, arylalkyl and heteroarylalkyl groups are optionally substituted with one to six halo;
or R¹ and R² can be taken together with the carbon atom to which they are attached to form a C₃₋₈ cycloalkyl ring which is optionally substituted with one to six halo;
D is aryl, heteroaryl, C₃₋₈ cycloalkyl or heterocyclyl wherein each said aryl, heteroaryl, cycloalkyl and heterocyclyl groups, which may be monocyclic or bicyclic, is optionally substituted on either the carbon or the heteroatom with one to five substituents independently selected from the group consisting of C₁₋₆ alkyl, haloalkyl, halo or cyano;
E is aryl, heteroaryl, C₃₋₈ cycloalkyl or heterocyclyl wherein each said aryl, heteroaryl, cycloalkyl and heterocyclyl groups, which may be monocyclic or bicyclic, is optionally substituted on either the carbon or the heteroatom with one to five substituents independently selected from the group consisting of C₁₋₆ alkyl, haloalkyl, halo or cyano;
R³ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR⁵, —C(O)OSi[CH(CH₃)₂]₃, —OR⁴, —OR⁵, —C(O)R⁵, —R⁵C(O)R⁴, —C(O)R⁴, —C(O)N(R^{a})(R^{b}), —C(O)N(R⁷)(R⁷), —C(O)N(R⁵)(R⁶), —C(R^{a})(R^{b})OH, —SR⁷, —SR⁴, —R⁵SR⁴, —R⁴, —C(R⁴)₃, —C(R⁵)(R⁶)N(R⁴)₂, —NR⁵C(O)NR⁵S(O)₂R⁴, —SO₂R⁵, —SO(R⁷), —SO₂R⁴, —SOₘN(R^{c})(R^{d}), —SOₘCH(R⁵)(R⁶), —SO₂N(R⁵)C(O)(R⁷), —SO₂(R⁵)C(O)N(R⁷)₂, —OSO₂R⁵, —N(R⁵)(R⁶), —N(R⁵)C(O)N(R⁵)(R⁴), —N(R⁵)C(O)R⁴, —N(R⁵)C(O)R⁵, —N(R⁵)C(O)OR⁵, —N(R⁵)SO₂(R⁵), —C(R⁵)(R⁶)NR⁵C(R⁵)(R⁶)R⁴, —C(R⁵)(R⁶)N(R⁵)R⁴, —C(R⁵)(R⁶)N(R⁵)(R⁶), —C(R⁵)(R⁶)SC(R⁵)(R⁶)(R⁴), R⁵S—, —C(R^{a})(R^{b})NR^{a}C(R^{a})(R^{b})(R⁴), —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁴, —C(O)C(R^{a})(R^{b})S(R^{a}), C(R^{a})(R^{b})C(O)N(R^{a})(R^{b}), C(R^{a})(R^{b})C(O)OH, —B(OH)₂, —OCH₂O— or 4,4,5,5-tetramethyl-1,3,2-dioxaborolan-2-yl; wherein said alkyl, alkenyl, alkynyl, alkyloxy, aryl, heteroaryl, cycloalkyl and heterocyclyl groups are optionally substituted on either the carbon or the heteroatom with one to five substituents independently selected from C₁₋₆ alkyl, halo, keto, cyano, haloalkyl, hydroxyalkyl, —OR⁴, —NO₂, —NH₂, —NHS(O)₂R⁵, —R⁴SO₂R⁷, —SO₂R⁷, —SO(R⁷), —SR⁷, —SR⁴, —SOₘN(R^{c})(R^{d}), —SOₘN(R⁵)C(O)(R⁷), —C(R⁵)(R⁶)N(R⁵)(R⁶), —C(R⁵)(R⁶)OH, —COOH, —C(R^{a})(R^{b})C(O)N(R^{a})(R^{b}), —C(O)(R^{a})(R^{b}), —C(O)NH₂, —C(O)NHR⁴, —N(R⁵)C(R⁵)(R⁶)(R⁴), —N(R⁵)CO(R⁴), —NH(CH₂)₂OH, —NHC(O)OR⁵, —Si(CH₃)₃, heterocycyl, aryl, or heteroaryl;
R⁴ is hydrogen, aryl, aryl(C₁₋₄) alkyl, heteroaryl, heteroaryl(C₁₋₄)alkyl, C₃₋₈cycloalkyl, C₃₋₈cycloalkyl(C₁₋₄)alkyl or heterocyclyl(C₁₋₄)alkyl wherein said groups are optionally substituted with one, two, or three substituents independently selected from halo, alkoxy or —SO₂R⁷;
R⁵ is hydrogen or C₁₋₆ alkyl;
R⁶ is hydrogen or C₁₋₆ alkyl;
R⁷ is hydrogen or C₁₋₆ alkyl which is optionally substituted with one, two, or three substituents independently selected from halo, alkoxy, cyano, —NR⁵ or —SR⁵;
R^{a} is hydrogen, C₁₋₆ alkyl, (C₁₋₆ alkyl)aryl, (C₁₋₆alkyl)hydroxyl, —O(C₁₋₆alkyl), hydroxyl, halo, aryl, heteroaryl, C₃₋₈ cycloalkyl or heterocyclyl, wherein said alkyl, aryl, heteroaryl, cycloalkyl and heterocyclyl groups are optionally substituted on either the carbon or the heteroatom with one, two, or three substituents independently selected from C₁₋₆ alkyl or halo;
R^{b} is hydrogen, C₁₋₆ alkyl, (C₁₋₆ alkyl)aryl, (C₁₋₆ alkyl)hydroxyl, —O(C₁₋₆alkyl), hydroxyl, halo, aryl, heteroaryl, C₃₋₈ cycloalkyl or heterocyclyl, wherein said alkyl, aryl, heteroaryl, cycloalkyl and heterocyclyl groups are optionally substituted on either the carbon or the heteroatom with one, two, or three substituents independently selected from C₁₋₆ alkyl or halo;
or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with one or two substituents independently selected from C₁₋₆ alkyl and halo;
each m is independently selected from an integer from zero to two;
or a pharmaceutically acceptable salt, stereoisomer or N-oxide derivative thereof.

In a class of the invention, X is OH or hydrogen.

In a class of the invention, D is aryl.

In a class of the invention, E is aryl or heteroaryl, wherein said aryl or heteroaryl group is optionally substituted on either the carbon or the heteroatom with one to five substituents independently selected from C₁₋₆ alkyl, haloalkyl or halo.

In a class of the invention, R¹ is hydrogen or C₁₋₆ alkyl which is optionally substituted with one to three fluoro. In a subclass of the invention, R¹ is hydrogen or C₁₋₃ alkyl.

In a class of the invention, R² is hydrogen or C₁₋₃ alkyl.

In a class of the invention, R³ is hydrogen, C₁₋₆ alkyl, C₃₋₈ cycloalkyl, —C(O)R⁵, —C(R^{a})(R^{b})OH, —SO₂R⁵, C(R^{a})(R^{b})C(O)N(R^{a})(R^{b}) or C(R^{a})(R^{b})C(O)OH, wherein said alkyl or cycloallyl groups are optionally substituted on either the carbon or the heteroatom with one to five substituents independently selected from C₁₋₆ alkyl, cyano, halo, C(O)NH₂, or —OR⁴. In a subclass of the invention, R³ is C₃₋₈ cycloalkyl which is optionally substituted with cyano. In a further subclass of the invention, R³ is cyclopropanecarbonitrile.

Reference to the preferred embodiments set forth above is meant to include all combinations of particular and preferred groups unless stated otherwise.

Specific embodiments of the present invention include, but are not limited to:
1-[4″-(1-amino-2,2-difluoroethyl)biphenyl-4-yl]cyclopropanecarboxamide;
1-{4′-[(1S)-1-amino-2,2-difluoroethyl]biphenyl-4-yl }-N-cyclopropylcyclopropanecarboxamide;
1-{4′-[(1S)-1-amino-2,2,2-trifluoroethyl]-2-fluorobiphenyl-4-yl}cyclopropanecarboxamide;
1-{2-fluoro-4′-[(1R)-2,2,2-trifluoro-1-hydroxyethyl]biphenyl-4-yl}cyclopropanecarboxamide;
1-{4′-[(1S)-2,2-difluoro-1-hydroxyethyl]biphenyl-4-yl}cyclopropanecarboxamide;
1-{4′-[(1R)-2,2-difluoro-1-hydroxyethyl]biphenyl-4-yl}cyclopropanecarboxamide;
1-[4′-(1-amino-2,2-difluoroethyl)-2-fluorobiphenyl-4-yl]cyclopropanecarboxamide;
2-{4′-[(1R)-2,2,2-trifluoro-1-hydroxyethyl]biphenyl-4-yl}propanoic acid;
(2S)-2-{4′-[(1R)-2,2,2-trifluoro-1-hydroxyethyl]biphenyl-4-yl}propanoic acid;
(2S)-2-{4′-[(1R)-2,2,2-trifluoro-1-hydroxyethyl]biphenyl-4-yl}propanamide;
1-[4′-(1-amino-2,2-difluoroethyl)biphenyl-4-yl]cyclopropanecarboxylic acid;
2-[4′-2,2-difluoro-1-hydroxyethyl)-2-fluorobiphenyl-4-yl]acetamide;
2,2-difluoro-1-[4-(4-methyl-1,3-thiazol-2-yl)phenyl]ethanol;
1-[4′-(2,2-difluoro-1-hydroxyethyl)biphenyl-4-yl]-2-methylpropan-2-ol;
1-{6-[4-(2,2-difluoro-1-hydroxyethyl)phenyl]pyridin-3-yl}cyclopropanol;
1-[4′(2,2-difluoro-1-hydroxyethyl)-2-fluorobiphenyl-4-yl]cyclopropanol;
(1R)-1-{4′-[(1S)-1-amino-2,2,2-trifluoroethyl]biphenyl-4-yl}-2,2-difluoroethanol;
2-[4′-(2,2-difluoro-1-hydroxyethyl)-2-fluorobiphenyl-4-yl]-2-methylpropanamide;
2-[4′(1-amino-2,2-difluoroethyl)-2-fluorobiphenyl-4-yl]-2-methylpropanamide;
2-{2-fluoro-4′-[(1R)-2,2,2-trifluoro-1-hydroxyethyl]biphenyl-4-yl}-2-methylpropanamide;
1-[4′-(2,2-difluoro-1-hydroxyethyl)biphenyl-4-yl]cyclopropanecarboxamide;
1-{4-[6-(2,2,2-trifluoro-1-hydroxyethyl)pyridin-3-yl]phenyl}cyclopropanecarboxamide;
1-(3-fluoro-4-[6-(2,2,2-trifluoro-1-hydroxyethyl)pyridin-3-yl]phenyl)cyclopropanecarboxamide;
1-biphenyl-4-yl-2,2,2-trifluoroethanol;
(1-biphenyl-4-yl-2,2,2-trifluoroethyl)amine;
2,2-difluoro-1-{4′-[(1R)-2,2,2-trifluoro-1-hydroxyethyl]biphenyl-4-yl}ethanone;
1-{4′-[(1S)-1-amino-2,2,2-trifluoroethyl]biphenyl-4-yl}-2,2-difluoroethanone;
1,1-difluoro-2-{4′-[(1R)-2,2,2-trifluoro-1-hydroxyethyl]biphenyl-4-yl}propan-2-ol;
2-{4′-[(1S)-1-amino-2,2,2-trifluoroethyl]biphenyl-4-yl}-1,1-difluoropropan-2-ol;
1-[4′-(2,2-difluoro-1-hydroxyethyl)-2-fluorobiphenyl-4-yl]cyclopropanecarbonitrile;
1-[4′-(2,2-difluoro-1-hydroxyethyl)-2-fluorobiphenyl-4-yl]cyclopropanecarboxamide
2,2-difluoro-1-[4′-(methylsulfonyl)biphenyl-4-yl]ethanol;
2,2,2-trifluoro-1-[4′-(methylsulfonyl)biphenyl-4-yl]ethane-1,1-diol;
1-(4-bromophenyl)-2,2-difluoroethanone;
N-cyclopropyl-1-[4′-(2,2-difluoro-1-hydroxyethyl)-2-fluorobiphenyl-4-yl]cyclopropanecarboxamide;
1-[4′-(1-amino-2,2-difluoroethyl)-2-fluorobiphenyl-4-yl]-N-cyclopropylcyclopropanecarboxamide;
1-(4-bromophenyl)-2,2-difluoroethanol;
1-{4′-[(1R)-1-amino-2,2,2-trifluoro-1-methylethyl]biphenyl-4-yl}cyclopropanecarboxamide;
1-[4′-(2,2,2-trifluoro-1-hydroxy-1-methylethyl)biphenyl-4-yl]cyclopropanecarboxamide;
1-{4′-[(2,4-difluorophenyl)(hydroxy)methyl]biphenyl-4-yl}cyclopropanecarboxamide;
1-{4′-[amino(2,4-difluorophenyl)methyl]biphenyl-4-yl}cyclopropanecarboxamide;
1-[4′-(2,2-difluoro-1-hydroxyethyl)-3′-fluorobiphenyl-4-yl]cyclopropanecarboxamide;
(1R)-1-[4′-(2,2-difluoro-1-hydroxyethyl)biphenyl-4-yl]-2,2,2-trifluoroethanol;
1-{4′-[(1S)-1-amino-2,2,2-trifluoroethyl]biphenyl-4-yl}-2,2-difluoroethanol;
{(1S)-2,2,2-trifluoro-1-[4′-(methylsulfonyl)biphenyl-4-yl]ethyl}amine;
1-{4′-[(1S)-1-amino-2,2,2-trifluoroethyl]biphenyl-4-yl}cyclopropanecarboxylic acid;
1-{4′-[(1S)-1-amino-2,2-difluoroethyl]biphenyl-4-yl}cyclopropanecarboxamide;
2-[4′-(1-amino-2,2,2-trifluoroethyl)biphenyl-4-yl]propanamide;
2-{4′-[(1R)-2,2,2-trifluoro-1-hydroxyethyl]biphenyl-4-yl}propanamide;
(2S)-2-{2-fluoro-4′-[(1R)-2,2,2-trifluoro-1-hydroxyethyl]biphenyl-4-yl}propanamide;
(2S)-2-{4′-[(1S)-1-amino-2,2,2-trifluoroethyl]-2-fluorobiphenyl-4-yl}propanamide;
(1R)-1-(4′-bromobiphenyl-4-yl)-2,2-difluoroethanol;
1-{4′-[(1R)-2,2-difluoro-1-hydroxyethyl]biphenyl-4-yl}-2,2,2-trifluoroethanone.
1-biphenyl-4-yl-2,2,2-trifluoroethanol;
(1-biphen-4-yl-2,2,2-trifluoroethyl)amine;
(1R)-1-(4′-bromobiphenyl-4-yl)-2,2-difluoroethanol;
1-{4′-[(1R)-2,2-difluoro-1-hydroxylethyl]biphenyl-4-yl}-2,2,2-trifluororethanone;
1-[2-fluoro-4′-(1-hydroxy-1-methylethyl)biphenyl-4-yl]cyclopropanecarbonitrile;
1-[2-fluoro-4′-(2,2,2-trifluoro-1-hydroxyethyl)biphenyl-4-yl]cyclopropanecarbonitrile;
1-[2-fluoro-4′-(trifluoroacetyl)biphenyl-4-yl]cyclopropanecarbonitrile;
1-{2-fluoro-4′-[2,2,2-trifluoro-1-hydroxy-1-(trifluoromethyl)ethyl]biphenyl-4-yl}cyclopropanecarbonitrile;
1-[4′-(2,2,2-trifluoro-1-hydroxyethyl)biphenyl-4-yl]cyclopropanecarbonitrile;
1-[4′-(2,2,2-trifluoro-1-hydroxyethyl)biphenyl-3-yl]cyclopropanecarbonitrile;
1-(2-fluoro-4′-isopropylbiphenyl-4-yl)cyclopropanecarbonitrile;
1-[2-fluoro-4′-(2-hydroxypiperidin-2-yl)biphenyl-4-yl]cyclopropanecarbonitrile;
1-[2-fluoro-4′-(1-hydroxycyclobutyl)biphenyl-4-yl]cyclopropanecarbonitrile;
2,2,2-trifluoro-1-(4′-isopropylbiphenyl-4-yl)ethanol;
1-[2 fluoro-4′-(2-hydroxy-2-methylpropyl)biphenyl-4-yl]cyclopropanecarbonitrile;
1-[2-fluoro-3′-(2-hydroxy-2-methylpropyl)biphenyl-4-yl]cyclopropanecarbonitrile;
1-[4-(1-benzothien-3-yl)-3-fluorophenyl]cyclopropanecarbonitrile;
1-[2-fluoro-3′-(1-hydroxy-1-methylethyl)biphenyl-4-yl]cyclopropanecarbonitrile;
1-{2-fluoro-2′-[hydroxy(phenyl)methyl]biphenyl-4-yl}cyclopropanecarbonitrile;
1-{2-fluoro-4′-[hydroxy(1,3-thiazol-2-yl)methyl]biphenyl-4-yl}cyclopropanecarbonitrile;
1-[2-fluoro-3′-(3-hydroxy-3-methyl-2-oxobutyl)biphenyl-4-yl]cyclopropanecarbonitrile;
1-{3-fluoro-4-[5-(1-hydroxy-1-methylethyl)pyridin-2-yl]phenyl}cyclopropanecarbonitrile;
1-[2-fluoro-4′-(hydroxymethyl)biphenyl-4-yl]cyclopropanecarbonitrile;
1-[2-fluoro-4′-(3-hydroxy-3-methylbutyl)biphenyl-4-yl]cyclopropanecarbonitrile;
1-[4-(5-acetyl-2-thienyl)-3-fluorophenyl]cyclopropanecarbonitrile;
1-{3-fluoro-4-[5-(methylsulfonyl)pyridin-2-yl]phenyl}cyclopropanecarbonitrile;
methyl 4′-(1-cyanocyclopropyl)-2′-fluorobiphenyl-4-carboxylate;
1-(4′-benzoyl-2-fluorobiphenyl-4-yl)cyclopropanecarbonitrile;
1-(3′-acetyl-2-fluorobiphenyl-4-yl)cyclopropanecarbonitrile;
1-(3′-ethyl-2-fluorobiphenyl-4-yl)cyclopropanecarbonitrile;
1-[2-fluoro-4′-(2-hydroxyethyl)biphenyl-4-yl]cyclopropanecarbonitrile;
1-[2-fluoro-4′-(1-hydroxyethyl)biphenyl-4-yl]cyclopropanecarbonitrile;
1-[2-fluoro-3′-(2-hydroxyethyl)biphenyl-4-yl]cyclopropanecarbonitrile;
1-(2-fluoro-1,1′:3′,1″-terphenyl-4-yl)cyclopropanecarbonitrile;
1-(2-fluoro-1,1′:2′,1″-terphenyl-4-yl)cyclopropanecarbonitrile;
1-(2-fluoro-1,1′:4′,1″-terphenyl-4-yl)cyclopropanecarbonitrile;
1-(2-fluorobiphenyl-4-yl)cyclopropanecarbonitrile;
1-(2-fluoro-3′-methylbiphenyl-4-yl)cyclopropanecarbonitrile;
1-(2-fluoro-2′-methylbiphenyl-4-yl)cyclopropanecarbonitrile;
1-(4′-ethyl-2-fluorobiphenyl-4-yl)cyclopropanecarbonitrile;
1-(2-fluoro-2′-isopropylbiphenyl-4-yl)cyclopropanecarbonitrile;
1-(2-fluoro-4′-methylbiphenyl-4-yl)cyclopropanecarbonitrile;
1-[3-fluoro-4-(2-naphthyl)phenyl]cyclopropanecarbonitrile;
1-(4′-acetyl-2-fluorobiphenyl-4-yl)cyclopropanecarbonitrile;
1-[3-fluoro-4-(1H-indol-5-yl)phenyl]cyclopropanecarbonitrile;
1,1′-(2,2′-difluorobiphenyl-4,4′-diyl)dicyclopropanecarbonitrile;
1-(2-fluoro-4′-pyridin-3-ylbiphenyl-4-yl)cyclopropanecarbonitrile;
1-(2-fluoro-4′-isopropylbiphenyl-4-yl)cyclopropanecarbonitrile;
1-[4′-(1-amino-1-methylethyl)-2-fluorobiphenyl-4-yl]cyclopropanecarbonitrile;
[4′-(1-hydroxy-1-methyl ethyl)biphenyl-4-yl]acetonitrile;
4′-(1-hydroxy-1-methylethyl)biphenyl-4-carboxamide;
4′-(1-hydroxy-1-methylethyl)biphenyl-4-sulfonamide;
4′-(1-hydroxy-1-methylethyl)biphenyl-3-carboxamide;
2-[4-(1-benzothien-3-yl)phenyl]propan-2-ol;
1-[4′-(1-hydroxy-1-methylethyl)biphenyl-3-yl]ethanone;
2-[4-(2-naphthyl)phenyl]propan-2-ol;
1-[4′-(1-hydroxy-1-methylethyl)biphenyl-4-yl]ethanone;
2-(1,1′:4′,1″-terphenyl-4-yl)propan-2-ol;
2-(1,1′:2′,1″-terphenyl-4-yl)propan-2-ol;
2-(1,1′:3′,1″-terphenyl-4-yl)propan-2-ol;
2-[4′-(methylsulfonyl)biphenyl-4-yl]propan-2-ol;
1-[4′-(1-hydroxy-1-methylethyl)biphenyl-3-yl]cyclopropanecarbonitrile;
2,2′-biphenyl-4,4′-diyldipropan-2-ol;
2-[3′-(methylsulfonyl)biphenyl-4-yl]propan-2-ol;
1-[2-fluoro-4′-(1-hydroxy-1-methylethyl)biphenyl-4-yl]cyclopropanecarboxylic acid;
2-{4′-[(methylsulfonyl)methyl]biphenyl-4-yl}propan-2-ol;
1-[4′-(1-hydroxy-1-methylethyl)biphenyl-4-yl]cyclopropanecarboxamide;
1-[2-fluoro-4′-(1-hydroxy-1-methylethyl)biphenyl-4-yl]methanesulfonamide;
1-{6-[4-(1-hydroxy-1-methylethyl)phenyl]pyridin-3-yl}cyclopropanecarbonitrile;
1-[4′-(1-hydroxy-1-methylethyl)biphenyl-3-yl]cyclopropanecarboxamide;
2-(4′-pyridin-3-ylbiphenyl-4-yl)propan-2-ol;
3-[4-(1-hydroxy-1-methylethyl)phenyl]quinoline-2-carbonitrile;
1-[4′-(1-hydroxy-1-methylethyl)biphenyl-4-yl]-N-methylcyclopropanecarboxamide;
[({1-[4′-(1-hydroxy-1-methylethyl)biphenyl-4-yl]cyclopropyl}carbonyl)(methylene)-λ⁵-azanyl]acetonitril
2-(4′-isopropoxybiphenyl-4-yl)propan-2-ol;
1-[2-fluoro-4′-(1-hydroxy-1-methylethyl)biphenyl-4-yl]cyclopropanecarboxamide;
   or a pharmaceutically acceptable salt, stereoisomer or N-oxide derivative thereof.

Also included within the scope of the present invention is a pharmaceutical composition which is comprised of a compound of Formula I as described above and a pharmaceutically acceptable carrier. The invention is also contemplated to encompass a pharmaceutical composition which is comprised of a pharmaceutically acceptable carrier and any of the compounds specifically disclosed in the present application, alone or in combination with any other disclosed compound. These and other aspects of the invention will be apparent from the teachings contained herein.

### Utilities

The compounds of the present invention are inhibitors of MAO-A and/or MAO-B and are therefore useful to treat or prevent neurological diseases or conditions in mamnals, preferably humans.

“Neurological diseases or conditions” refers to abnormalities of neurotransmitter synthesis, storage, release, or degradation or changes in the number and affinity of receptors which can affect neurotransmission and cause clinical disorders. Neurological diseases or conditions includes, but is not limited to, mood disorders, depression, bipolar disorders, substance-induced mood disorders, anxiety disorders, cognitive disorders, delirium, amnestic disorders, Alzheimer's disease, schizophrenia, schizophreniform disorder, schizoaffective disorder, delusional disorder, brief psychotic disorder, shared psychotic disorder, addictive behaviors, movement disorders, akinesias, akinetic-rigid syndromes, Parkinson's disease, medication-induced parkinsonism, Gilles de la Tourette's syndrome, epilepsy, dyskinesias, chorea, myoclonus, tics, dystonia, obesity, bulimia nervosa, compulsive eating disorders, eating disorders associated with excessive food intake, osteoarthritis, repetitive motion pain, dental pain, cancer pain, myofascial pain, perioperative pain, chronic pain, neuropathic pain, post-traumatic pain, trigeminal neuralgia, migraine, attention-deficit hyperactivity disorder, conduct disorder, muscular spasms, urinary incontinence, amyotrophic lateral sclerosis, neuronal damage, ocular damage, retinopathy, macular degeneration of the eye, hearing loss, tinnitus, emesis, brain edema or sleep disorders.

An embodiment of the invention is a method of inhibiting MAO-A activity in a mammal in need thereof, comprising administering to the mammal a therapeutically effective amount of any of the compounds or any of the pharmaceutical compositions described above.

Another embodiment of the invention is a method of inhibiting MAO-B activity in a mammal in need thereof, comprising administering to the mammal a therapeutically effective amount of any of the compounds or any of the pharmaceutical compositions described above.

Another embodiment of the invention is a method of inhibiting MAO-A and/or B activity in a mammal in need thereof, comprising administering to the mammal a therapeutically effective amount of any of the compounds or any of the pharmaceutical compositions described above.

Another embodiment of the invention is a method of treating or preventing mood disorders, depression, bipolar disorders, substance-induced mood disorders, anxiety disorders, cognitive disorders, delirium, amnestic disorders, Alzheimer's disease, schizophrenia, schizophreniform disorder, schizoaffective disorder, delusional disorder, brief psychotic disorder, shared psychotic disorder, addictive behaviors, movement disorders, akinesias, akinetic-rigid syndromes, Parkinson's disease, medication-induced parkinsonism, Gilles de la Tourette's syndrome, epilepsy, dyskinesias, chorea, myoclonus, tics, dystonia, obesity, bulimia nervosa, compulsive eating disorders, eating disorders associated with excessive food intake, osteoarthritis, repetitive motion pain, dental pain, cancer pain, myofascial pain, perioperative pain, chronic pain, neuropathic pain, post-traumatic pain, trigeminal neuralgia, migraine, attention-deficit hyperactivity disorder, conduct disorder, muscular spasms, urinary incontinence, amyotrophic lateral sclerosis, neuronal damage, ocular damage, retinopathy, macular degeneration of the eye, hearing loss, tinnitus, emesis, brain edema or sleep disorders in a mammal in need thereof, comprising administering to the mammal a therapeutically effective amount of any of the compounds or any of the pharmaceutical compositions described above.

Another embodiment of the invention is a method of treating depression in a mammal in need thereof, comprising administering to the mammal a therapeutically effective amount of any of the compounds or any of the pharmaceutical compositions described above. The utility of MAO inhibitors in the treatment of depression is known in the literature, see, Liebowitz M R, et al., “Reversible and irreversible monoamine oxidase inhibitors in other psychiatric disorders.” Acta Psychiatr Scand Suppl. 1990; 360:29-34.

Another embodiment of the invention is a method of treating anxiety in a mammal in need thereof, comprising administering to the mammal a therapeutically effective amount of any of the compounds or any of the pharmaceutical compositions described above. The utility of MAO inhibitors in the treatment of anxiety is known in the literature, see, Galynker I, et al., “Low-Dose Risperidone and Quetiapine as Monotherapy for Comorbid Anxiety and Depression.” J Clin Psychiatry. 2005 April; 66(4):544.

Another embodiment of the invention is a method of treating substance induced mood disorders in a mammal in need thereof, comprising administering to the mammal a therapeutically effective amount of any of the compounds or any of the pharmaceutical compositions described above. The utility of MAO inhibitors in the treatment of substance induced mood disorders is known in the literature, see, Takahashi S, et al., “Monoamine oxidase activity in blood platelets in alcoholism.” Folia Psychiatr Neurol Jpn. 1976; 30(4):455-62.

Another embodiment of the invention is a method of treating delirium and delusional disorder in a mammal in need thereof, comprising administering to the mammal a therapeutically effective amount of any of the compounds or any of the pharmaceutical compositions described above. The utility of MAO inhibitors in the treatment of delirium and delusional disorder is known in the literature, see, C. L. DeVane and J. Mintzer, “Risperidone in the management of psychiatric and neurodegenerative disease in the elderly: an update.” Psychopharmacol Bull. 2003; 37(4): 116-32.

Another embodiment of the invention is a method of treating amnestic disorder in a mammal in need thereof, comprising administering to the mammal a therapeutically effective amount of any of the compounds or any of the pharmaceutical compositions described above. The utility of MAO inhibitors in the treatment of amnestic disorders is known in the literature, see, Purdon, S. E. et al., “Neuropsychological change in early phase schizophrenia during 12 months of treatment with olanzapine, risperidone, or haloperidol,” Arch. Gen. Psychiatry 57 (2000), pp. 249-258.

Another embodiment of the invention is a method of treating Alzheimer's disease in a mammal in need thereof, comprising administering to the mammal a therapeutically effective amount of any of the compounds or any of the pharmaceutical compositions described above. The utility of MAO inhibitors in the treatment of Alzheimer's disease is known in the literature, see, Ono, K. et al., “Anti-Parkinsonian agenst have anti-amyloidogenic activity for Alzheimer's beta-amyloid fibrils in vitro,” Neurochem Int. 2006 March; 48(4):275-85.

Another embodiment of the invention is a method of treating epilepsy in a mammal in need thereof, comprising administering to the mammal a therapeutically effective amount of any of the compounds or any of the pharmaceutical compositions described above. The utility of MAO inhibitors in the treatment of seizures is known in the literature, see, Jobe A, et al., “Three children with a syndrome of obesity and overgrowth, atypical psychosis, and seizures: a problem in neuropsychopharmacology.” J Child Neurol. 2000 August; 15(8):518-28. Another embodiment of the invention is a method of treating Parkinson's disease in a mammal in need thereof, comprising administering to the mammal a therapeutically effective amount of any of the compounds or any of the pharmaceutical compositions described above. The utility of MAO inhibitors in the treatment of Parkinsonf's disease is known in the literature, see, Weinstock, et al., “A novel cholinesterdas and brain-selective monoamine oxidase inhibitor for the treatment of dementia comorbid with depression and Parkinson's disease. Prog. Neuropsychopharmacol. Biol. Psychiatry 27 (2003), pp. 555-561.

Another embodiment of the invention is a method of treating pain in a mammal in need thereof, comprising administering to the mammal a therapeutically effective amount of any of the compounds or any of the pharmaceutical compositions described above. Pain includes repetitive motion pain, dental pain, cancer pain, myofascial pain, perioperative pain, chronic pain, neuropathic pain, post-traumatic pain, trigeminal neuralgia and migraine. The utility of MAO inhibitors in the treatment of pain is known in the literature, see, Pirildar S, et al., “A preliminary open-label study of moclobemide treatment of pain disorder.” Psychopharmacol Bull. 2003 Summer; 37(3): 127-34; Silberstein, S D, et al., “Preventive treatment of migraine: an overview.” Cephalalgia. 1997 April; 17(2):67-72.

Another embodiment of the invention is a method of treating attention-deficit hyperactivity disorder in a mammal in need thereof, comprising administering to the mammal a therapeutically effective amount of any of the compounds or any of the pharmaceutical compositions described above. The utility of MAO inhibitors in the treatment of attention-deficit hyperactivity disorder is known in the literature, see, Spencer T J., “ADHD treatment across the life cycle.” J Clin Psychiatry. 2004; 65 Suppl 3:22-6.

Another embodiment of the invention is a method of treating eating disorders in a mammal in need thereof, comprising administering to the mammal a therapeutically effective amount of any of the compounds or any of the pharmaceutical compositions described above. The utility of MAO inhibitors in the treatment of eating disorders, bulimia nervosa is known in the literature, see, AS. Kaplan, “Academy for Eating Disorders International Conference on Eating Disorders. Denver, Colo., USA, May 29-31, 2003.” Expert Opin Investig Drugs. 2003 August; 12(8):1441-3.

Another embodiment of the invention is a method of treating sleep disorders in a mammal in need thereof, comprising administering to the mammal a therapeutically effective amount of any of the compounds or any of the pharmaceutical compositions described above. The utility of MAO inhibitors in the treatment of sleep disorders, is known in the literature, see, Hublin, C., et al., “Selegiline in the treatment of narcolepsy.” Neurology 44: 2095-2101; Louden, MB, et al., “Activation of selegiline (1-deprenyl) of REM sleep behaviour disorder in parkinsonism.” West Virg Med J 91: 101.

Another embodiment of the invention is a method of treating mood disorders in a mammal in need thereof, comprising administering to the mammal a therapeutically effective amount of any of the compounds or any of the pharmaceutical compositions described above. The utility of MAO inhibitors in the treatment of mood disorders, including bipolar disorders, is known in the literature, see, Gutierrez B, et al., “Association analysis between a functional polymorphism in the monoamine oxidase A gene promoter and severe mood disorders.” Psychiatr Genet. 2004 December; 14(4):203-8.

Another embodiment of the invention is a method of treating cognitive disorders in a mammal in need thereof, comprising administering to the mammal a therapeutically effective amount of any of the compounds or any of the pharmaceutical compositions described above. The utility of MAO inhibitors in the treatment of cognitive disorders, is known in the literature, see, Schneider L S., “New therapeutic approaches to cognitive impairment.” 3 Clin Psychiatry. 1998; 59 Suppl 11:8-13.

Another embodiment of the invention is a method of treating schizophrenia in a mammal in need thereof, comprising administering to the mammal a therapeutically effective amount of any of the compounds or any of the pharmaceutical compositions described above. The utility of MAO inhibitors in the treatment of schizophrenia, including schizophreniform disorder and schizoaffective disorder, is known in the literature, see, Toren, P., et al., “Benefit-risk assessment of atypical antipsychotics in the treatment of schizophrenia and comorbid disorders in children and adolescents.” Drug Saf. 2004; 27(14): 1135-56.

Another embodiment of the invention is a method of treating movement disorders in a mammal in need thereof, comprising administering to the mammal a therapeutically effective amount of any of the compounds or any of the pharmaceutical compositions described above. The utility of MAO inhibitors in the treatment of movement disorders, including dyskinesias, dystonia and akinesia, is known in the literature, see, Waters C., “Other pharmacological treatments for motor complications and dyskinesias.” Mov Disord. 2005 May; 20 Suppl 11:S3844; Pearce, J K, et al., “The monoamine reuptake blocker brasofensine reverses akinesia without dyskinesia in MPTP-treated and levodopa-primed common marmosets.” Mov Disord. 2002 September; 17(5):877-86.

Another embodiment of the invention is a method of treating hearing loss in a mammal in need thereof, comprising administering to the mammal a therapeutically effective amount of any of the compounds or any of the pharmaceutical compositions described above. The utility of MAO inhibitors in the treatment of hearing loss, including tinnitus, is known in the literature, see, Sharpe M H, “Auditory attention in early Parkinson's disease: an impairment in focused attention.” Neuropsychologia. 1992 January; 30(1): 101-6.

Another embodiment of the invention is a method of treating brain edema in a mammal in need thereof, comprising administering to the mammal a therapeutically effective amount of any of the compounds or any of the pharmaceutical compositions described above. The utility of MAO inhibitors in the treatment of brain edema is known in the literature, see, Huang W, “Neuroprotective effect of rasagiline, a selective monoamine oxidase-B inhibitor, against closed head injury in the mouse” Eur J Pharmacol. 1999 Feb. 5; 366(2-3):127-35.

Another embodiment of the invention is a method of treating neuronal damage in a mammal in need thereof, comprising administering to the mammal a therapeutically effective amount of any of the compounds or any of the pharmaceutical compositions described above. The utility of MAO inhibitors in the treatment of neuronal damage is known in the literature, see, Mandel S, et al., “Mechanism of neuroprotective action of the anti-Parkinson drug rasagiline and its derivatives.” Brain Res Brain Res Rev. 2005 April; 48(2):379-87.

Another embodiment of the invention is a method of treating amyotrophic lateral sclerosis in a mammal in need thereof, comprising administering to the mammal a therapeutically effective amount of any of the compounds or any of the pharmaceutical compositions described above. The utility of MAO inhibitors in the treatment of amyotrophic lateral sclerosis is known in the literature, see, Orru, S., “Association of monoamine oxidase B alleles with age at onset in amyotrophic lateral sclerosis.” Neuromuscul Disord. 1999 December; 9(8):593-7.

Another embodiment of the invention is a method of treating conduct disorder in a mammal in need thereof, comprising administering to the mammal a therapeutically effective amount of any of the compounds or any of the pharmaceutical compositions described above. The utility of MAO inhibitors in the treatment of conduct disorder is known in the literature, see, Haberstick, BC., “Monoamine oxidase A (MAOA) and antisocial behaviors in the presence of childhood and adolescent maltreatment.” Am J Med Genet B Neuropsychiatr Genet. 2005 May 5; 135(1):59-64.

Another embodiment of the invention is a method of treating ocular damage in a mammal in need thereof, comprising administering to the mammal a therapeutically effective amount of any of the compounds or any of the pharmaceutical compositions described above. The utility of MAO inhibitors in the treatment of ocular damage, including retinopathy and macular degeneration of the eye, is known in the literature, see, Xu L, et al., “1-Deprenyl, blocking apoptosis and regulating gene expression in cultured retinal neurons.” Biochem Pharmacol. 1999 Oct. 1; 58(7): 1183-90.

Another embodiment of the invention is a method of treating myoclonus, Gilles de la Tourette's syndrome, dystonia and tics in a mammal in need thereof, comprising administering to the mammal a therapeutically effective amount of any of the compounds or any of the pharmaceutical compositions described above. The utility of MAO inhibitors in the treatment of myoclonus, Gilles de la Tourette's syndrome, dystonia and tics is known in the literature, see, J. Jankovic and J. Beach J., “Long-term effects of tetrabenazine in hyperkinetic movement disorders.” Neurology. 1997 February; 48(2):358-62.

Another embodiment of the invention is a method of treating obesity in a mammal in need thereof, comprising administering to the mammal a therapeutically effective amount of any of the compounds or any of the pharmaceutical compositions described above. The utility of MAO inhibitors in the treatment of obesity is known in the literature, see, Visentin V, et al., “Alteration of amine oxidase activity in the adipose tissue of obese subjects.” Obes Res. 2004 March; 12(3):547-55

Another embodiment of the invention is a method of treating osteoarthritis in a mammal in need thereof, comprising administering to the mammal a therapeutically effective amount of any of the compounds or any of the pharmaceutical compositions described above. The utility of MAO inhibitors in the treatment of osteoarthritis is known in the literature, see, Chambers M G, et al., “Chondrocytic monoamine oxidase activity in the development of natural murine osteoarhritis.” Int J Exp Pathol. 1992 April; 73(2): 115-23.

Another embodiment of the invention is a method of treating chorea in a mammal in need thereof, comprising administering to the mammal a therapeutically effective amount of any of the compounds or any of the pharmaceutical compositions described above. The utility of MAO inhibitors in the treatment of chorea is known in the literature, see, J. Mann and E. Chiu, “Platelet monoamine oxidase activity in Huntington's chorea.” J Neurol Neurosurg Psychiatry. 1978 September; 41(9):809-12.

Exemplifying the invention is the use of a pharmaceutical composition comprising a compound as described herein for the manufacture of a medicament for the treatment of mood disorders, depression, bipolar disorders, substance-induced mood disorders, anxiety disorders, cognitive disorders, delirium, amnestic disorders, Alzheimer's disease, schizophrenia, schizophreniform disorder, schizoaffective disorder, delusional disorder, brief psychotic disorder, shared psychotic disorder, addictive behaviors, movement disorders, akinesias, akinetic-rigid syndromes, Parkinson's disease, medication-induced parkinsonism, Gilles de la Tourette's syndrome, epilepsy, dyskinesias, chorea, myoclonus, tics, dystonia, obesity, bulimia nervosa, compulsive eating disorders, eating disorders associated with excessive food intake, osteoarthritis, repetitive motion pain, dental pain, cancer pain, myofascial pain, perioperative pain, chronic pain, neuropathic pain, post-traumatic pain, trigeminal neuralgia, migraine, attention-deficit hyperactivity disorder, conduct disorder, muscular spasms, urinary incontinence, amyotrophic lateral sclerosis, neuronal damage, ocular damage, retinopathy, macular degeneration of the eye, hearing loss, tinnitus, emesis, brain edema or sleep disorders in a mammal in need thereof.

The compounds of this invention may be administered to mammals, preferably humans, either alone or, preferably, in combination with pharmaceutically acceptable carriers or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and topical routes of administration.

In the case of tablets for oral use, carriers which are commonly used include lactose and corn starch, and lubricating agents, such as magnesium stearate, are commonly added. For oral administration in capsule form, useful diluents include lactose and dried corn starch. For oral use of a therapeutic compound according to this invention, the selected compound may be administered, for example, in the form of tablets or capsules, or as an aqueous solution or suspension. For oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. When aqueous suspensions are required for oral use, the active ingredient is combined with emulsifying and suspending agents. If desired, certain sweetening or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The instant compounds are also useful in combination with known agents useful for treating or preventing mood disorders, depression, bipolar disorders, substance-induced mood disorders, anxiety disorders, cognitive disorders, delirium, amnestic disorders, Alzheimer's disease, schizophrenia, schizophreniform disorder, schizoaffective disorder, delusional disorder, brief psychotic disorder, shared psychotic disorder, addictive behaviors, movement disorders, akinesias, akinetic-rigid syndromes, Parkinson's disease, medication-induced parkinsonism, Gilles de la Tourette's syndrome, epilepsy, dyskinesias, chorea, myoclonus, tics, dystonia, obesity, bulimia nervosa, compulsive eating disorders, eating disorders associated with excessive food intake, osteoarthritis, repetitive motion pain, dental pain, cancer pain, myofascial pain, perioperative pain, chronic pain, neuropathic pain, post-traumatic pain, trigeminal neuralgia, migraine, attention-deficit hyperactivity disorder, conduct disorder, muscular spasms, urinary incontinence, amyotrophic lateral sclerosis, neuronal damage, ocular damage, retinopathy, macular degeneration of the eye, hearing loss, tinnitus, emesis, brain edema or sleep disorders. Combinations of the presently disclosed compounds with other agents useful in treating or preventing neurological conditions are within the scope of the invention. A person of ordinary skill in the art would be able to discern which combinations of agents would be useful based on the particular characteristics of the drugs and the disease involved. Such agents include the following: an anti-depressant, an anti-anxiety agent, an anti-Alzheimer's agent, a sedative, a hypnotic, an anxiolytic, an antipsychotic, a cyclopyrrolone, an imidazopyridine, a pyrazolopyrimidine, a minor tranquilizer, a melatonin agonist, a melatonin antagonist, a melatonergic agent, a benzodiazepine, a barbiturate, a 5HT-2 antagonist, levodopa, an anticholinergic, a trihexyphenidyl hydrochloride, a COMT inhibitor, an antioxidant, an A2a adenosine receptor antagonist, a cholinergic agonist, a NMDA receptor antagonist, a serotonin receptor antagonist, a monoamine oxidase inhibitor, a dopamine receptor agonist, a neuroleptic agent, an anoretic agent, a selective serotonin reuptake inhibitor, a halogenated amphetamine derivative, an opiate agonist, a lipoxygenase inhibitor, an interleukin inhibitor, an NMDA antagonist, an inhibitor of nitric oxide, a non-steroidal antiinflammatory agent, a cytokine-suppressing antiinflammatory agent, a pain reliever, a potentiator, an H2-antagonist, simethicone, aluminum hydroxide, magnesium hydroxide, a decongestant, an antitussive, and an antihistamine.

Exemplifying the invention is a pharmaceutical composition comprising a compound as described herein and another agent selected from: an anti-depressant, an anti-anxiety agent, an anti-Alzheimer's agent, a sedative, a hypnotic, an anxiolytic, an antipsychotic, a cyclopyrrolone, an imidazopyridine, a pyrazolopyrimidine, a minor tranquilizer, a melatonin agonist, a melatonin antagonist, a melatonergic agent, a benzodiazepine, a barbiturate, a 5HT-2 antagonist, levodopa, an anticholinergic, a trihexyphenidyl hydrochloride, a COMT inhibitor, an antioxidant, an A2a adenosine receptor antagonist, a cholinergic agonist, a NMDA receptor antagonist, a serotonin receptor antagonist, a monoamine oxidase inhibitor, a dopamine receptor agonist, a neuroleptic agent, an anoretic agent, a selective serotonin reuptake inhibitor, a halogenated amphetamine derivative, an opiate agonist, a lipoxygenase inhibitor, an interleukin inhibitor, an NMDA antagonist, an inhibitor of nitric oxide, a non-steroidal antiinflammatory agent, a cytokine-suppressing antiinflammatory agent, a pain reliever, a potentiator, an H2-antagonist, simethicone, aluminum hydroxide, magnesium hydroxide, a decongestant, an antitussive, and an antihistamine.

Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be co-administered, either in concomitant therapy or in a fixed combination. The following list of combinations is illustrative only and not intended to be limiting in any way.

In one embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In another embodiment, the subject compound may be employed in combination with anti-Alzheimer's agents; beta-secretase inhibitors; gamma-secretase inhibitors; HMG-CoA reductase inhibitors; NSAID's including ibuprofen; vitamin E; anti-amyloid antibodies; CB-1 receptor antagonists or CB-1 receptor inverse agonists; antibiotics such as doxycycline and rifampin; N-methyl-D-aspartate (NMDA) receptor antagonists, such as memantine; cholinesterase inhibitors such as galantamine, rivastigmine, donepezil, and tacrine; growth hormone secretagogues such as ibutamoren, ibutamoren mesylate, and capromorelin; histamine H₃ antagonists; AMPA agonists; PDE IV inhibitors; GABA_{A} inverse agonists; or neuronal nicotinic agonists.

In another embodiment, the subject compound may be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, clonazepam, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, and combinations thereof, and the like, or the subject compound may be administered in conjunction with the use of physical methods such as with light therapy or electrical stimulation.

In another embodiment, the subject compound may be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol)hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In another embodiment, the subject compound may be employed in combination with acetophenazine, alentemol, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, risperidone, sulpiride, tetrabenazine, trihexyphenidyl, thioridazine, thiothixene or trifluoperazine.

In another embodiment, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with thesubject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form.

In another embodiment, the subject compound may be employed in combination with an anoretic agent such as aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, metharnphetamine, norpseudoephedrine, pentorex, phendimetrazine, phenmetrazine, phentermine, phenylpropanolamine, picilorex and sibutramine; selective serotonin reuptake inhibitor (SSRI); halogenated amphetamine derivatives, including chlorphentermine, cloforex, clortermine, dexfenfluramine, fenfluramine, picilorex and sibutramine; and pharmaceutically acceptable salts thereof.

In another embodiment, the subject compound may be employed in combination with an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, asprin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the subject compound may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antitussive such as codeine, hydrocodone, caramiphen, carbetapentane, or dextramethorphan; a diuretic; and a sedating or non-sedating antihistamine.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents. The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

The present invention also encompasses a pharmaceutical composition useful in the treatment of neurological conditions, comprising the administration of a therapeutically effective amount of the compounds of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for a cathepsin dependent condition. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/lkg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The compounds of the present invention can be used in combination with other agents useful for treating neurological conditions. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating neurological conditions includes in principle any combination with any pharmaceutical composition useful for treating disorders related to neurological functioning.

The scope of the invention therefore encompasses the use of the instantly claimed compounds in combination with a second agent selected from: an anti-depressant, an anti-anxiety agent, an anti-Alzheimer's agent, a sedative, a hypnotic, an anxiolytic, an antipsychotic, a cyclopyrrolone, an imidazopyridine, a pyrazolopyrimidine, a minor tranquilizer, a melatonin agonist, a melatonin antagonist, a melatonergic agent, a benzodiazepine, a barbiturate, a 5HT-2 antagonist, levodopa, an anticholinergic, a trihexyphenidyl hydrochloride, a COMT inhibitor, an antioxidant, an A2a adenosine receptor antagonist, a cholinergic agonist, a NMDA receptor antagonist, a serotonin receptor antagonist, a monoamine oxidase inhibitor, a dopamine receptor agonist, a neuroleptic agent, an anoretic agent, a selective serotonin reuptake inhibitor, a halogenated amphetamine derivative, an opiate agonist, a lipoxygenase inhibitor, an interleukin inhibitor, an NMDA antagonist, an inhibitor of nitric oxide, a non-steroidal antiinflammatory agent, a cytokine-suppressing antiinflammatory agent, a pain reliever, a potentiator, an H2-antagonist, simethicone, aluminum hydroxide, magnesium hydroxide, a decongestant, an antitussive, and an antihistamineand the pharmaceutically acceptable salts and mixtures thereof.

These and other aspects of the invention will be apparent from the teachings contained herein.

### DEFINITIONS

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

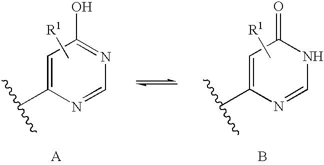

When any variable (e.g. R1, R², Ra etc.) occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring carbon atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having one to ten carbon atoms unless otherwise specified. For example, C₁-C₁₀, as in “C₁-C₁₀ alkyl” is defined to include groups having 1, 2, 3, 4, 5, 6, 7, 8, 9 or 10 carbons in a linear, branched, or cyclic arrangement. For example, “C₁-C₁₀ alkyl” specifically includes methyl, ethyl, propyl, butyl, pentyl, hexyl, heptyl, octyl, nonyl, decyl, and so on.

“Alkoxy” or “alkyloxy” represents an alkyl group as defined above, unless otherwise indicated, wherein said alkyl group is attached through an oxygen bridge. Examples of alkoxy include methoxy, ethoxy and the like.

The term “cycloalkyl” shall mean cyclic rings of alkanes of three to eight total carbon atoms, unless otherwise indicated, or any number within this range (i.e., cyclopropyl, cyclobutyl, cyclopentyl, cyclohexyl, cycloheptyl or cyclooctyl).

If no number of carbon atoms is specified, the term “alkenyl” refers to a non-aromatic hydrocarbon radical, straight or branched, containing from 2 to 10 carbon atoms and at least 1 carbon to carbon double bond. Preferably 1 carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “alkynyl” shall mean a substituting univalent group derived by conceptual removal of one hydrogen atom from a straight or branched-chain acyclic unsaturated hydrocarbon containing at least one triple bond (i.e., —C≡CH, —CH₂C≡CH, —C≡CCH₃, —CH₂C≡CCH₂(CH₃)₂, etc.).

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, “aryl” is intended to mean any stable monocyclic or bicyclic carbon ring of up to 12 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or tricyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, pyranyl, pyrazinyl, pyrazolyl, pyriclazinyl, pyridopyridinyl, pyridyl, pyrimidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, methylenedioxybenzene, benzothiazolyl, benzothienyl, quinolinyl, isoquinolinyl, oxazolyl, and tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo” or “halogen” as used herein is intended to include chloro, fluoro, bromo and iodo. The term “keto” means carbonyl (C═O).

The term “haloalkyl” means an alkyl radical as defined above, unless otherwise specified, that is substituted with one to five, preferably one to three halogen. Representative examples include, but are not limited to trifluoromethyl, dichloroethyl, and the like.

The term “arylalkyl” includes an alkyl portion where alkyl is as defined above and to include an aryl portion where aryl is as defined above. Examples of arylalkyl include, but are not limited to, benzyl, fluorobenzyl, chlorobenzyl, phenylethyl, phenylpropyl, fluorophenylethyl, and chlorophenylethyl. Examples of alkylaryl include, but are not limited to, toluoyl, ethylphenyl, and propylphenyl.

The term “heteroarylalkyl” as used herein, shall refer to a system that includes a heteroaryl portion, where heteroaryl is as defined above, and contains an alkyl portion. Examples of heteroarylalkyl include, but are not limited to, thienylmethyl, thienylethyl, thienylpropyl, pyridylmethyl, pyridylethyl and imidazoylmethyl.

The term “cycloalkylalkyl” includes an alkyl portion where alkyl is as defined above and also includes a cycloalkyl portion where cycloalkyl is as defined above. Examples of cycloalkylalkyl include, but are not limited to, cyclopropylmethyl, cyclopentylmethyl, cyclohexylmethyl, cyclopropylethyl, and the like.

The term “heterocycloalkylalkyl” or “heterocyclylalkyl” includes an alkyl portion where alkyl is as defined above and also includes a heterocycloalkyl portion where heterocycloalkyl is as defined above. Examples of heterocycloalkylalkyl include, but are not limited to, morpholinylmethyl, piperazinylmethyl, pyrrolidinylmethyl, and the like.

The term “hydroxyalkyl” or “alkylhydroxyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, and the like.

The term “heterocycle” or “heterocyclyl” as used herein is intended to mean a 5- to 10-membered nonaromatic ring, unless otherwise specified, containing from 1 to 4 heteroatoms selected from the group consisting of O, N, S, SO, or SO₂ and includes bicyclic groups. “Heterocyclyl” therefore includes, but is not limited to the following: piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. Also when compounds of Formula I contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

Whenever the term “alkyl” or “aryl” or either of their prefix roots appear in a name of a substituent (e.g., aryl C₀₋₈ alkyl) it shall be interpreted as including those limitations given above for “alkyl” and “aryl.” Designated numbers of carbon atoms (e.g., C₁₋₁₀) shall refer independently to the number of carbon atoms in an alkyl or cyclic alkyl moiety or to the alkyl portion of a larger substituent in which alkyl appears as its prefix root.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared front organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm Sci., 1977:66:1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

For purposes of this specification, the following abbreviations have the indicated meanings:
AcOH=acetic acid
BH₃.Me₂S=borane-methyl sulfide complex
Boc=t-butyloxycarbonyl
Boc₂O=di-tert-butyl dicarbonate
BuLi=butyl lithium
CCl₄=carbon tetrachloride
CH₂Cl₂=methylene chloride
CH₃CN=acetonitrile
CHCl₃=chloroform
Cs₂CO₃=cesium carbonate
CuI=copper iodide
DIAD=diisopropyl azodicarboxylate
DIP-Cl=B-chlorodiisopinocampheylborane
DMA=N,N-dimethyl acetamide
DMAP=4-(dimethylamino)pyridine
DMF=N,N-dimethylformamide
DMSO=dimethylsulfoxide
DPPA=diphenylphosphoryl azide
EDCl=1-(3-dimethylaminopropyl)-3-ethylcarbodiimide hydrochloride
Et₂O=diethyl ether
Et₃N=triethylamine
EtOAc=ethyl acetate
EtOH=ethanol
HATU=o-7-azabenzotriazol-1-yl)-N,N,N′,N′-tetramethyluronium hexafluorophosphate HOAc=acetic acid
K₂CO₃=potassium carbonate
KOBu^{t}=potassium tert-butoxide
LiOH=lithium hydroxide
mCPBA=metachloroperbenzoic acid
MeOH methanol
MeSO₃H=methane sulfonic acid
MgSO₄=magnesium sulfate
Ms=methanesulfonyl=mesyl
MsCl=methanesulfonyl chloride
MTBE=methyl tert-butyl ether
NaBH₄=sodium borohydride
NaH=sodium hydride
Na₂CO₃=sodium carbonate
NaHCO₃=sodium hydrogencarbonate
NaOH=sodium hydroxide
Na₂SO₄=sodium sulfate
NBS=N-bromosuccinimide
NH₃=ammonia
NH₄Cl=ammonium chloride
Pd/C=palladium on carbon
PdCl₂(dppf)=[1,1′-bis(diphenylphosphino)ferrocene]dichloropalladium(II)
Pd₂(dba)₃=tris(dibenzylideneacetone)dipalladium(0)
PG=protecting group
PPh₃=triphenylphosphine
PPTS pyridinium p-toluenesulfonate
iPr₂Nli=lithium diisopropyl amide
PyBOP=benzotriazol-1-yloxytris(pyrrolidino)phosphonium-hexafluorophosphate
rt=room temperature
sat. aq.=saturated aqueous
TFA=trifluoroacetic acid
THF=tetrahydrofuran
tlc=thin layer chromatography
Me=methyl
Et=ethyl
n-Pr=normal propyl
i-Pr=isopropyl
n-Bu=normal butyl
i-Bu=isobutyl
s-Bu=secondary butyl
t-Bu=tertiary butyl

The novel compounds of the present invention can be prepared according to the following general procedures using appropriate materials and are further exemplified by the following specific examples. The compounds illustrated in the examples are not, however, to be construed as forming the only genus that is considered as the invention. The following examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

### Schemes

Compounds of the present invention can be prepared according to Scheme 1, as indicated below. Thus a dihalo aromatic compound can be mono-lithiated and reacted with either ethyl difluoroacetate or ethyl trifluoroacetate to generate the difluoroketone or trifluoroketone, respectively. Reduction of the ketone with sodium borohydride provides the alcohol. This reduction can also be performed enantioselectively with chiral reagents such as alpine borane or B-chlorodiisopinocampheylborane. If the substituent on D system is a halogen, a palladium-catalyzed Suzuki coupling with an appropriate boronic acid provides compounds of the current invention. Alternatively, the difluoroketone or trifluoroketone can be converted to the corresponding primary amine by first forming the N-trimethylsilyl-ketimine followed by in situ reduction with BH₃.Me₂S. The reduction of the imine can also be performed enantioselectively with B-butyl-diphenylpyrrolidino-oxazaborolidine. The primary amine can be further elaborated with a palladium-catalyzed Suzuki coupling with an appropriate boronic acid to provide compounds of the current invention.

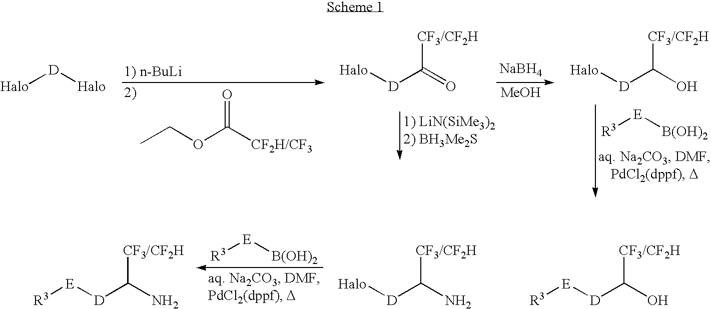

Compounds of the present invention may also be prepared according to Scheme 2, as indicated below. Thus a dihalo aromatic compound can be mono-lithiated and reacted with an aryl or heteroarylaldehyde to generate the corresponding alcohol. If the substituent on the D system of the resultant alcohol is a halogen, a palladium-atalyzed Suzuki coupling with an appropriate boronic acid provides compounds of the current invention. Alternatively, the alcohol can be converted to the corresponding primary amine via a mitsonobu reaction with azide and reduction of the azide to the amine using conditions such as 1,3-propanedithiol and triethylamine. The amine can then be converted to compounds of the present invention by elaborating the D ring via a Suzuki coupling.

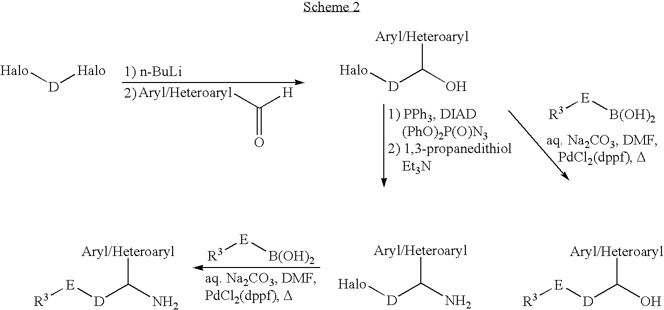

Compounds of the present invention may also be prepared according to Scheme 3, as indicated below. A dihalo aromatic compound can be mono-lithiated and reacted with acetone or hexafluoroacetone to generate the corresponding tertiary alcohol. This alcohol can be converted into compounds of the current invention by the method described in Scheme 3.

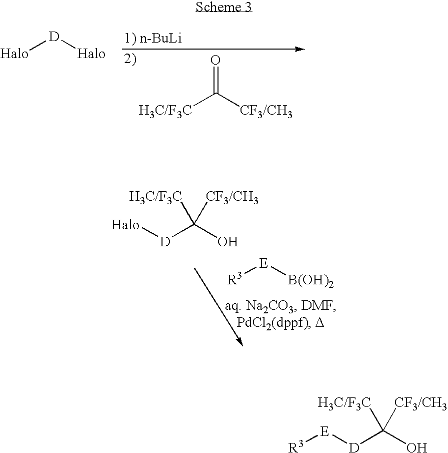

Compounds of the current invention may also be prepared according to Scheme 4. The difluoroketone or trifluoroketone generated in Scheme 1 can be converted to the corresponding tertiary alcohol by reaction with an alkyllithium. This alcohol can then be elaborated to compounds of the current invention via a Suzuki coupling reaction.

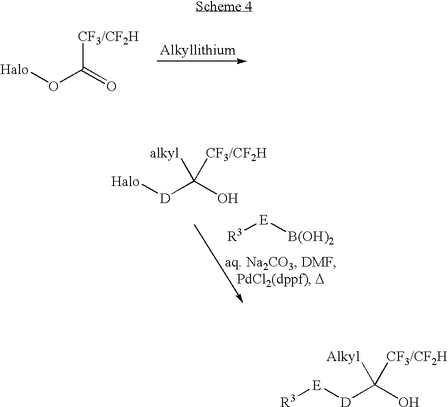

The following examples describe the synthesis of selected compounds of the present invention and are included for illustrative purposes and do not limit the scope of the invention in any way.

### EXAMPLE 1

### Synthesis of 1-[4′-(2,2-difluoro-1-hydroxyethyl)-2-fluorobiphenyl-4-yl]cyclopropanecarbonitrile

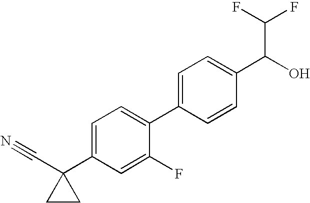

### Step 1: Synthesis of 1-(4-bromophenyl)-2,2-difluoroethanol

To a cold (0° C.) solution of 1-(4-bromophenyl)-2,2-difluoroethanone (1.07 g, 4.55 mmol) in MeOH (30 mL) was added NaBH (260 mg, 6.83 mmol) portionwise. The reaction was warmed to rt and stirred for 16 h. Acetone (5 mL) was added followed by water (10 mL). The mixture was concentrated under reduced pressure followed by the addition of Et₂O (50 mL) and 0.1 N HCl (25 mL). The layers were separated and the organic extract was washed with brine (1×50 mL), dried (MgSO₄) and concentrated to yield the title compound.

### Step 2: Synthesis of 2,2-difluoro-1-[4-(4,4,5,5-tetramethyl-1,3,2-dioxaborolan-2-yl)phenyl]ethanol

A solution of 1-(4-bromophenyl)-2,2-difluoroethanol (1.07 g, 4.55 mmol), potassium acetate (1.33 g, 13.5 mmol) and bis(pinacolato)diboron (1.37 g, 5.4 mmol) in DMF (20 mL) was bubbled with nitrogen for 5 minutes then PdCl₂dppf (184 m g, 0.225 mmol) was added and the reaction mixture was stirred at 80° C. overnight. The reaction mixture was concentrated under reduced pressure and filtered through a plug of silica gel to remove any insoluables. The filtrate was concentrated to afford the title compound which was used as such in Step 7.

¹H NMR δ (ppm)(Acetone): 7.75 (2H, m), 7.48 (2H, d), 5.90 (1H, dt), 5.30 (1H, d), 4.87 (1H, m), 1.31 (12H, s).

### Step 3: Preparation of 1-bromo-4-(bromomethyl)-2-fluorobenzene

To a room temperature solution of 4-bromo-3-fluorotoluene (10.6 g) in 150 mL of carbon tetrachloride were added benzoyl peroxide (100 mg) and N-bromosuccinimide (10 g). The mixture was heated at 80° C. (with shine light) for 4 hours. The reaction mixture was cooled to 0° C. and filtered through celite, washed with hexanes and the solvent removed in vacuo. The crude material was purified by chromatography on SiO₂ using hexanes to yield the title compound containing ˜30% of 1-bromo-4-(dibromomethyl)-2-fluorobenzene as impurity.

¹H NMR (CD₃COCD₃) δ7.66-7.10 (1H, m), 7.42 (1H, d), 7.29 (1H, d), 4.66 (2H, s).

### Step 4: Preparation of (4-bromo-3-fluorophenyl)methanol

To a room temperature solution of 1-bromo-4-(bromomethyl)-2-fluorobenzene from Step 3 (11.8 g) in DMF (150 mL) was added sodium acetate (10.8 g). The mixture was heated at 80° C. for 16 hours. It was cooled to room temperature and poured into ice and saturated aqueous sodium bicarbonate (200 mL), and extracted with diethyl ether (2×100 mL). The combined extracts were washed with brine, dried with magnesium sulfate and the solvent removed in vacuo. The crude material was purified by chromatography on SiO₂ using ethyl acetate and hexanes (1:25 to 1:10) to yield 4-bromo-3-fluorobenzyl acetate (containing about 15% of 4-bromo-3-fluorobenzaldehyde). The residue was dissolved in methanol (100 mL), cooled to 0° C. and sodium methoxide (250 mg) was added. The reaction mixture was stirred at room temperature for 2 hours. It was cooled to 0° C. and sodium borohydride was added (1.5 g). The mixture was stirred at 0° C. for 1 hour and poured into ice and saturated aqueous ammonium chloride (200 mL). The mixture was extracted with ethyl acetate (2×100 mL). The combined extracts were washed with brine, dried with magnesium sulfate and the solvent removed in vacuo. The residue was purified by chromatography on SiO₂ using ethyl acetate and hexanes (1:5 to 1:3) to yield the title compound.

¹H N NMR (CD₃COCD₃) δ 7.55-7.65 (1H, m), 7.28 (1H, d), 7.15 (1H, d), 4.63 (2H, d), 4.50 (1H, t).

### Step 5: Preparation of (4-bromo-3-fluorophenyl)acetonitrile

To a −78° C. solution of (4-bromo-3-fluorophenyl)methanol from Step 4 (7.2 g) and triethylamine (5.9 mL) in dichloromethane (300 mL) was slowly added methanesulphonyl chloride (3.0 mL). The reaction mixture was stirred at 0° C. for 1 hour and then poured into ice and saturated aqueous ammonium chloride and partitioned. The aqueous layer was extracted with dichloromethane (1×150 mL). The combined extracts were washed with brine, dried with magnesium sulfate and the solvent removed in vacuo. The residue was dissolved in DMF (150 mL) and sodium cyanide (5.1 g) was added. The reaction mixture was stirred at room temperature for 2 hours and poured into ice and water (100 mL). The aqueous phase was extracted with ethyl acetate (2×100 mL). The combined organic extracts were washed with brine, dried with magnesium sulfate and the solvent removed in vacuo. The residue was purified by chromatography on SiO₂ using ethyl acetate and hexanes (1:10 to 1:5) to yield the title compound.

¹H NMR (CD₃COCD₃) δ 7.70-7.78 (1H, m), 7.39 (1H, d), 7.28 (1H, d), 4.09 (2H, s).

### Step 6: Preparation of 1-(4-bromo-3-fluorophenyl)cyclopropanecarbonitrile

To a room temperature solution of (4-bromo-3-fluorophenyl)acetonitrile from Step 5 (6.4 g) in a solution of 7.5 mL of sodium hydroxide (50% in water W/W) were added 1-bromo-2-chloroethane (4.0 mL) and benzyltriethylammonium chloride (204 mg). The mixture was heated at 60° C. for 5 hours. The reaction mixture was cooled to room temperature and poured into water (100 mL). The aqueous phase was extracted with ethyl acetate (200 mL). The combined organic extracts were washed with water (100 mL), hydrogen chloride (100 mL, 10% HCl in water) and brine and then dried with magnesium sulfate and the solvent removed in vacuo. The residue was purified by swish using methyl t-butyl ether and hexanes to yield the title compound.

¹H NMR (CD₃COCD₃) δ 7.69-7.73 (1H, m), 7.28 (1H, d), 7.25 (1H, d), 1.80-1.87 (2H, m), 1.59-1.65 (2H, m).

### Step 7: Synthesis of 1-[4′-(2,2-difluoro-1-hydroxyethyl)-2-fluorobiphenyl-4-yl]cyclopropanecarbonitrile

To a solution of 1-(4-bromo-3-fluorophenyl)tyclopropanecarbonitrile from step 6 (72 mg, 0.30 mmol) and 2,2-difluoro-1-[4-(4,4,5,5-tetramethyl-1,3,2-dioxaborolan-2-yl)phenyl]ethanol from step 2 (85 mg, 0.30 mmol) in DMF (3 mL) was added a solution of 2.0 M sodium carbonate (440 μL, 0.89 mmol). The mixture was bubbled with nitrogen for 10 minutes and then PdCl₂ddpf (12 mg, 0.015 mmol) was added. The solution was heated in the microwave at 120° C. for 500 seconds. Et₂O (50 mL) and water (50 mL) were added and the layers were separated. The aqueous phase was extracted with Et₂O (3×50 mL) and the combined organic extracts were washed with brine (1×50 mL), dried (Na₂SO₄) and concentrated. The resulting residue was flash chromatographed (50:50 EtOAc/Hexanes) to afford the title compound.

¹H NMR δ (ppm)(Acetone): 7.59 (5H, m), 7.35 (1H, d), 7.19 (1H, d), 5.97 (1H, dt), 4.92 (1H, dt), 1.82 (2H, m), 1.62 (2H, m).

### EXAMPLE 2

### Synthesis of 2-{4′-[(1R)-2,2,2-trifluoro-1-hydroxyethyllbiphen]-4-yl}propanamide

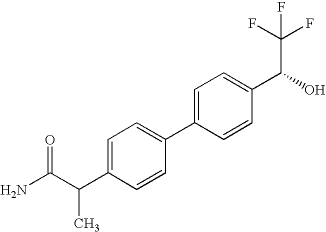

### Step 1: Synthesis of (1R)-1-(4-bromophenyl)-2,2,2-trifluoroethanol

1-(4-Bromophenyl)-2,2,2-trifluoroethanone was reduced enantioselectively with (−)DIP-Cl to afford the title compound as reported in Tetrahedron Asymmetry 1994, 1075.

### Step 2: Preparation of 2-(4-bromophenyl)propanoic acid

To a solution of 4-bromophenylacetic acid (60 g), 1,3-Dimethyl-3,4,5,6-tetrahydro-2(1H)-pyrimidinone (60.5 mL) and iodomethane (18 mL) in THF (900 mL) at −20° C. was added dropwise lithium bis(trimethylsilyl)amide (1M in THF, 586 mL) over 30 minutes. The reaction mixture was stirred at −20° C. for 2 h and warmed up to room temperature over 2 h and finally stirred at room temperature for 2 h. ¹H NMR of an aliquot showed 50% conversion. The reaction mixture was cooled to −20° C. and lithium bis(trimethylsilyl)amide (1M in THF, 140 mL) was added. The mixture was warmed up to room temperature over 2 h and aged overnight at room temperature. ¹H NMR of an aliquot showed 75% conversion. The reaction mixture was poured into ice and 6N HCl (190 mL), partitioned and extracted with ether (2×400 mL). The combined organic extracts were washed with a saturated NaCl solution, dried (MgSO₄) and concentrated under vacuum to yield the title compound. ¹H NMR showed about 20% of 4-bromophenylacetic acid. It was used as such in Step 3.

¹H NMR of title compound (CD₃COCD₃) δ 7.52 (2H, d), 7.32 (2H, d), 3.79 (1H, q), 1.46 (3H, d). Peaks of starting material not listed.

### Step 3 2-(4-bromophenyl)propanamide

To a solution of 2-(4-bromophenyl)propanoic acid (4.0 g, 17.5 mmol) in chloroform (175 mL) at 0° C. was added triethylamine (3.9 mL, 27.7 mmol) followed by isobutyl chloroformate (3.4 mL, 25.9 mmol). The reaction mixture was stirred at 0° C. for 2 h. Then ammonia gas was bubbled into the reaction mixture for 15 minutes and warmed up to room temperature for 1 h. The reaction mixture was poured to onto ice/water and extracted with CH₂Cl₂ (2×150 mL). The combined organic layers were washed with a saturated NaCl solution, dried (MgSO₄) and concentrated under vacuum followed by swish in MTBE/Hexanes to afford the title compound as colorless powder. ¹H NMR (CD₃COCD₃) δ 7.50 (2H, d), 7.35 (2H, d), 6.83 (1H, s), 6.22 (1H, s), 3.69 (1H, q), 1.40 (3H, d).

### Step 4: Preparation of 2-[4-(4,4,5,5-tetramethyl-1,3,2-dioxaborolan-2-yl)phenyl]propanamide

A solution of 2-(4-bromophenyl)propanamide (500 mg, 2.192 mmol), potassium acetate (753 mg, 7.67 mmol) and bis(pinacolato)diboron, 98% (724 mg, 2.85 mmol) in DMF (20 mL) was bubbled with nitrogen for 15 minutes then PdCl₂dppf (90 mg, 0.11 mmol) was added and bubbled again with nitrogen for 10 minutes. The reaction mixture was stirred at 65° C. overnight, poured onto ice/water and extracted with 50% EtOAc/Hexanes (3×50 mL). The combined organic layers were washed with a saturated NaCl solution, dried (MgSO₄) and concentrated under vacuum. The residue was purified by chromatography on silica gel (EtOAc/Hexane, 1:1 to 2:1) to afford the title compound.

¹H NMR (CD₃COCD₃) 7.70 (2H, d), 7.40 (2H, d), 6.75 (1H, s), 6.21 (1H, s), 3.70 (1H, q), 1.41 (3H, d), 1.34 (12H, s).

### Step 5: Synthesis of 2-{4′-[(1R)-2,2,2-trifluoro-1-hydroxyethyl]biphenyl-4-yl}propanamide

To a solution of 2-[4-(4,4,5,5-tetramethyl-1,3,2-dioxaborolan-2-yl)phenyl]propanamide from Step 4 (140 mg, 0.51 mmol) and (1R)-1-(4-bromophenyl)-2,2,2-trifluoroethanol from Step 1 (156 mg, 0.61 mmol) in DMF (6 mL) was added a solution of 2.0 M sodium carbonate (650 μL, 1.3 mmol). The mixture was bubbled with nitrogen for 10 minutes then PdCl₂ddpf (25 mg, 0.031 mmol) was added. The mixture was bubbled again with nitrogen for 10 minutes. Then the reaction mixture was stirred at 80° C. for 2 h, poured onto an ice/saturated NaHCO₃ solution and extracted with EtOAc (3×50 mL). The combined organic layers were washed with a saturated NaCl solution, dried (MgSO₄) and concentrated under vacuum. Purification by chromatography on silica gel using automatized gradiant pump system CombiFlash (EtOAc/Hexane, 50:50 to 80:20 for 30 minutes) afforded the title compound as pale yellow powder.

¹H NMR (500 MHz, Acetone): δ 7.72 (d, J=8.4 Hz, 2H); 7.65 (dd, J=3.2, 8.2 Hz, 4H); 7.49 (d, J=8.2 Hz, 2H); 6.80 (s, 1H); 6.20 (s, 1H); 5.90 (d, J=5.5 Hz, 1H); 5.32-5.26 (m, 1H); 3.75 (q, J=7.0 Hz, 1H); 1.46 (d, J=7.1 Hz, 3H). MS (+APCI); 324.2.

### EXAMPLE 3

### Synthesis of 1-{4′-[(1S)-1-amino-2,2-difluoroethyl]biphenyl-4-yl}cyclopropanecarboxamide

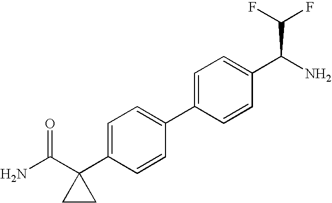

### Step 1: Synthesis of [(1S)-1-(4-bromophenyl)-2,2-difluoroethyl]amine

The title compound was synthesized starting from 1-(4-bromophenyl)-2,2-difluoroethanone as described in the synthesis of (1S)-1-(4-bromophenyl)-2,2,2-trifluoroethanamine (example 8)

### Step 2: Preparation of 1-(4-bromophenyl)cyclopropanecarbonitrile

To a room temperature solution of 4-bromophenylacetonitrile (18.0 g) in a solution of 22 mL of sodium hydroxide (50% in water W/W) were added 1-bromo-2-chloroethane and (12.0 mL) and benzyltriethylammonium chloride (627 mg). The mixture was heated at 60° C. overnight. The reaction mixture was cooled to room temperature and diethyl ether was added (300 mL) and partitioned. The ether layer was washed with water (100 mL), hydrogen chloride (100 mL, 10% HCl in water), brine and dried with magnesium sulfate. Upon removal of the solvent in vacuo, the residue was purified by swish using diethyl ether and hexanes to yield the title compound.

¹H NMR (CD₃COCD₃) δ 7.60 (2H, d), 7.35 (2H, d), 1.74-1.80 (2H, m), 1.52-1.57 (2H, m).

### Step 3: Preparation of 1-(4-bromophenyl)cyclopropanecarboxylic acid

To a room temperature solution of 1-(4-bromophenyl)cyclopropanecarbonitrile from Step 2 (13 g) in ethyl alcohol (110 mL) was added a solution of 56 mL of sodium hydroxide (25% NaOH in water W/W). The mixture was heated at 100° C. overnight. It was cooled to room temperature and poured into ice and hydrogen chloride (1 N), and extracted with dichloromethane (2×100 mL). The combined extracts were washed with brine, dried with magnesium sulfate and the solvent removed in vacuo to yield the title compound.

¹H NMR (CD₃COCD₃) δ 7.50 (2H, d), 7.35 (2H, d), 1.53-1.60 (2 μm), 1.18-1.22 (2H, m).

### Step 4: Preparation of 1-(4-bromophenyl)cyclopropanecarboxamide

To a −15° C. solution of 14-bromophenyl)cyclopropanecarboxylic acid from Step 3 (1.5 g) in chloroform (60 mL) were slowly added isobutyl chloroformate (900 μL) and triethylamine (1.1 mL). The reaction mixture was stirred at −15° C. for 2 hours. Then it was saturated with ammonia gas and stirred at −15° C. for 10 minutes. The reaction mixture was allowed to stand at room temperature for 1 hour then poured into water (60 mL) and partitioned. The aqueous layer was extracted with dichloromethane (2×60 mL). The combined extracts were washed with brine, dried with magnesium sulfate and the solvent removed in vacuo. The residue was purified by swish using diethyl ether and hexanes to yield the title compound.

¹H NMR (CD₃COCD₃) δ 7.54 (2H, d), 7.40 (2H, d), 6.45 (1H, bs), 5.96 (1H, bs), 1.42-1.48 (2H, m), 0.98-1.02 (2H, m).

### Step 5: Synthesis of 1-[4-(4,4,5,5-tetramethyl-1,3,2-dioxaborolan-2-yl)phenyl]cyclopropanecarboxamide

A solution of 14-bromophenyl)cyclopropanecarboxamide (9.4 g, 39.2 mmol), potassium acetate (13.45 g, 137 mmol) and bis(pinacolato)diboron, 98% (12.95 g, 51 mmol, 1.3 eq) in DMF (150 mL) was bubbled with nitrogen for 15 minutes then PdCl₂dppf (1.6 g, 1.959 mmol) was added and bubbled again with nitrogen for 10 minutes. The reaction mixture was stirred at 65° C. overnight, poured unto ice/water and extracted with 50% EtOAc/Hexanes (3×300 mL). The combined organic layers were washed with a saturated NaCl solution, dried (MgSO₄) and concentrated under vacuum. The residue was purified by chromatography on silica gel (EtOAc/Hexane, 1:1 to 2:1) followed by trituration with MTBE/Hexanes to afford the title compound as light beige solid powder.

¹H NMR δ (ppm)(Acetone): 7.74 (2H, d, J=8.0 Hz), 7.46 (2H, d, J=8.0 Hz), 6.34 (1H, s), 5.80 (1H, s), 1.48-1.46 (2H, m), 1.36 (12H, s), 1.00 (2H, q, J=3.4 Hz).

### Step 6: Synthesis of 1-{4′-[(1S)-1-amino-2,2-difluoroethyl]biphenyl-4-yl}cyclopropanecarboxamide

To a solution of [(1S)-1-(4-bromophenyl)-2,2-difluoroethyl]amine from Step 1 (2.1 g, 8.9 mmol) and 1-[4-(4,4,5,5-tetramethyl-1,3,2-dioxaborolan-2-yl)phenyl]cyclopropanecarboxamide from Step 5 (3.0 g, 10.45 mmol) in DMF (40 mL) was added a solution of 2.0 M sodium carbonate (11 mL, 22 mmol). The mixture was bubbled with nitrogen for 10 minutes then PdCl₂dppf (400 mg, 0.49 mmol) was added. The mixture was bubbled again with nitrogen for 10 minutes. Then the reaction mixture was stirred at 80° C. for 2 h, poured into an icy saturated NaHCO₃ solution and extracted with EtOAc (3×150 mL). The combined organic layers was washed with a saturated NaCl solution, dried (MgSO₄) and concentrated under vacuum. The residue was purified by chromatography on silica gel (EtOAc/Hexanes, 60:40 to 75:25 then 10% EtOH/EtOAc) followed by trituration in MTBE/hexanes to afford the title compound as pale yellow powder.

¹H NMR δ (ppm)(CD₃OD): 7.68 (4H, dd, J=2.3, 8.3 Hz), 7.53 (4H, dd, J=3.6, 8.2 Hz), 6.05-5.81 (1H, m), 4.23-4.17 (1H, m), 1.56 (2H, q, J=3.5 Hz), 1.15 (2H, q, J=3.5 Hz). MS (+APCI)=317.0. E.E.=100% (determined by Chiralcel OD 4.6×250 mm, 80% Hexanes/20% EtOH/0.1% diethylamine, Rt=19.447 minutes).

Optical rotation=+11.3 (c=1, MeOH)

### EXAMPLE 4

### Synthesis of 1-{4′-[(2,4-difluorophenyl)(hydroxy)methyl]biphenyl-4-yl}cyclopropanecarboxamide

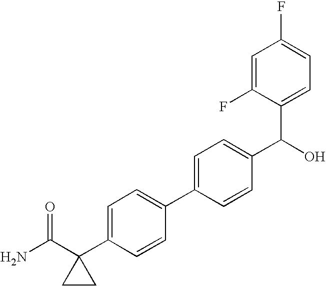

### Step 1: Synthesis of (4-bromophenyl)(2,4-difluorophenyl)methanol

To a cold (−78° C.), stirred solution of 1,4-dibromophenyl (12.3 g, 52 mmol) in Et₂O (200 mL) was added n-BuLi (2.5 M in hexanes, 19.2 mL, 48 mmol) and the reaction was stirred for 1 h. This mixture was then added to a cold (−78° C.) solution of 2,4-difluorobenzaldehyde (4.4 mL, 40 mmol) and stirred at −78° C. for 2 h. Water was added (200 mL) and the layers were separated. The aqueous layer was extracted with Et₂O (2×250 mL) and the combined organic extracts were dried (Na₂SO₄) and concentrated. The resulting residue was flash chromatographed (15:85 EtOAc/Hexanes) to afford the title compound.

### Step 2: Synthesis of 1-{4′-[(2,4-difluorophenyl)(hydroxy)methyl]biphenyl-4-yl}cyclopropanecarboxamide

To a solution of (4-bromophenyl)(2,4-difluorophenyl)methanol from Step 1 (90 mg, 0.3 mmol), 1-[4-(4,4,5,5-tetramethyl-1,3,2-dioxaborolan-2-yl)phenyl]cyclopropanecarboxamide from Step 5, Example 3 (170 mg, 0.36 mmol) and PdCl₂(dppf) (12 mg, 0.015 mmol) in dry DMF (4 mL) was added aqueous Na₂CO₃ (2 M, 450 μL, 0.9 mmol). The solution was heated at 80° C. for 16 h. The reaction mixture was cooled to room temperature followed by the addition of 1 M NaOH (5 mL) and Et₂O (50 mL). The layers were separated and the organic phase was washed with 1 N NaOH (25 mL), dried (Na₂SO₄) and concentrated. The resulting residue was flash chromatographed (70:30 EtOAc/Hexanes) to afford the title compound.

¹H NMR δ (ppm)(Acetone): 7.65 (4H, m), 7.50 (4H, m), 7.05 (1H, t), 6.95 (1H, t), 6.70 (1H, bs), 6.15 (1H, s), 5.95 (1H, bs), 1.49 (2H, m), 1.05 (2H, m).

### EXAMPLE 5

### Synthesis of 1-{4′-[amino(2,4-difluorophenyl)methyl]biphenyl-4-yl}cyclopropanecarboxamide

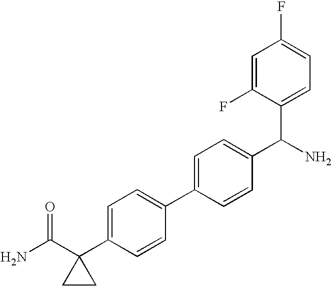

### Step 1: Synthesis of (4-bromophenyl)(2,4-difluorophenyl)methyl azide

To a solution of (4-bromophenyl)(2,4-difluorophenyl)methanol from Step 1, Example 4 (1.65 g, 5.5 mmol) and PPh₃ (1.73 g, 6.6 mmol) in dry THF (30 mL) was added DIAD (1.3 mL, 6.6 mmol) followed by DPPA (1.45 mL, 6.6 mmol). The reaction was stirred at rt for 5 h at which time water (50 mL) was added. The layers were separated and the aqueous phase was extracted with Et₂O (2×100 mL). The combined organic extracts were dried (Na₂SO₄) and concentrated. The resulting residue was flash chromatographed (5:95 EtOAc/Hexanes) to afford the title compound.

### Step 2: Synthesis of [(4-bromophenyl)(2,4-difluorophenyl)methyl]amine

To a solution of (4-bromophenyl)(2,4-difluorophenyl)methyl azide (1.16 g, 3.58 mmol) in MeOH (15 mL) was added 1,3-propanedithiol (1.1 mL, 10.7 mmol) followed by Et₃N (1.5 mL, 10.7 mmol). The reaction was stirred at rt for 4 h and then concentrated. The resulting residue was flash chromatographed (50:50 EtOAc/Hexanes to 70:30 EtOAc/Hexanes) to afford the title compound.

### Step 3: Synthesis of 1-{4′-[amino(2,4-difluorophenyl)methyl]biphenyl-4-yl}cyclopropanecarboxamide

To a solution of [(4-bromophenyl)(2,4-difluorophenyl)methyl]amine from Step 2 (90 mg, 0.3 mmol), 1-[4-(4,4,5,5-tetramethyl-1,3,2-dioxaborolan-2-yl)phenyl]cyclopropanecarboxamide from Step 5, Example 3 (170 mg, 0.36 mmol) and PdCl₂(dppf) (12 mg, 0.015 mmol) in dry DMF (4 mL) was added aqueous Na₂CO₃ (2 M, 450 μL, 0.9 mmol). The solution was heated at 80° C. for 16 h. The reaction mixture was cooled to room temperature followed by the addition of 1 M NaOH (5 mL) and Et₂O (50 mL). The layers were separated and the organic phase was washed with 1 N NaOH (25 mL), dried (Na₂SO₄) and concentrated. The resulting residue was flash chromatographed (95:5 EtOAc/Hexanes) to afford the title compound. ¹H NMR δ (ppm)(Acetone): 7.60 (4H, m), 7.51 (4H, m), 6.98 (2H, m), 6.60 (1H, bs), 6.02 (1H, s), 5.92 (1H, bs), 1.49 (2H, m), 1.03 (2H, m).

### EXAMPLE 6

### Synthesis of 1-[4′-(2,2,2-trifluoro-1-hydroxy-1-methylethyl)biphenyl-4-yl]cyclopropanecarboxamide

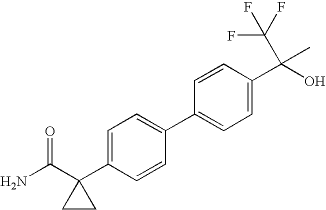

### Step 1: Synthesis of 1-[4′-4-(trifluoroacetyl)biphenyl-4-yl]cyclopropanecarboxamide

To a solution of 1-(4-bromophenyl)-2,2,2-trifluoroethanone (133 mg, 0.53 mmol), 1-[4-(4,4,5,5-tetramethyl-1,3,2-dioxaborolan-2-yl)phenyl]cyclopropanecarboxamide from Step 5, Example 3 (151 mg, 0.53 mmol) and PdCl₂(dppf) (43 mg, 0.053 mmol) in dry DMF (4 mL) was added aqueous Na₂CO₃ (2 M, 0.8 mL, 1.58 mmol). The solution was heated in the microwave at 120° C. for 500 seconds. Et₂O (50 mL) and water (50 mL) were added and the layers were separated. The aqueous phase was extracted with Et₂O (3×50 μL) and the combined organic extracts were washed with brine (1×50 mL), dried (Na₂SO₄) and concentrated. The resulting residue was flash chromatographed (50:50 EtOAc/Hexanes) to afford the title compound.

¹H NMR δ (ppm)(Acetone): 8.25 (2H, d), 8.03 (2H, d), 7.83 (2H, d), 7.62 (2H, d), 6.47 (1H, s), 5.96 (1H, s), 1.5 (2H, m), 1.07 (2H, m).

### Step 2: Synthesis of 1-[4′-(2,2,2-trifluoro-1-hydroxy-1-methylethyl)biphenyl-4-yl]cyclopropanecarboxamide

To a cold (−78° C.) solution of 1-[4′-(trifluoroacetyl)biphenyl-4-yl]cyclopropanecarboxamide from Step 1 (89 mg, 0.27 mmol) in THF (3 mL) was added MeLi (1.6 M in Et₂O, 340 μL, 0.54 mmol) and stirred for 2 h. Saturated aq. NH₄Cl (10 mL) was added and the layers were separated. The aqueous phase was extracted with EtOAc (3×50 mL) and the combined organic extracts were washed with brine (1×50 mL), dried (Na₂SO₄) and concentrated. The ¹H NMR of the residue indicated 50:50 starting material to product. Hence, the residue was redissolved in THF (3 mL), cooled to −78° C. and an additional aliquot of MeLi (1.3 mL, 2 mmol) was added. The reaction was stirred at −78° C. for 1 h, warmed to 0° C. for 10 min and then to rt for 10 min. Saturated aq. NH₄Cl (10 mL) was added and the layers were separated. The aqueous phase was extracted with EtOAc (3×50 mL) and the combined organic extracts were washed with brine (1×50 mL), dried (Na₂SO₄) and concentrated. The resulting residue was flash chromatographed (60:40 EtOAc/Hexanes) to afford the title compound.

¹H NMR δ (ppm)(Acetone): 7.80-7.65 (6H, m), 7.50 (2H, d), 6.40 (1H, s), 5.85 (1H, s), 1.80 (3H, s), 1.45 (2H, m), 1.02 (2H, m).

### EXAMPLE 7

### Synthesis of 1-{4-[6-(2,2,2-trifluoro-1-hydroxyethyl) yridine-3-yl]phenyl}cyclopropanecarboxamide

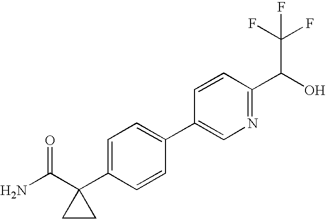

### Step 1: Synthesis of 1-(5-bromopyridin-2-yl)-2,2,2-trifluoroethanone

To a cold (−78° C.) solution of 2,5-dibromopyridine (8.0 g, 33.9 mmol) in toluene (230 mL) was added n-BuLi (2.5 M in hexanes, 13.5 mL, 33.8 mol) and the mixture was stirred for 30 min. 2,2,2-Trifluoroethyl trifluoroacetate (5.0 mL, 37.3 mmol) was added dropwise over 15 min and then the mixture was stirred at −78° C. for 2 h. The mixture was allowed to warm to room temperature and then quenched with sat. aq. NH₄Cl (50 mL). The layers were separated and the aqueous phase was extracted with EtOAc (3×100 mL). The combined organic extracts were dried (MgSO₄) and concentrated. The residue was subjected to flash column chromatography (gradient elution: 15:85 EtOAc:hexanes to 40:60 EtOAc:hexanes) to afford the title compound.

### Step 2: Synthesis of 1-(5-bromopyridin-2-yl)-2,2,2-trifluoroethanol

To a cold (0° C.) solution of 15-bromopyridin-2-yl)-2,2,2-trifluoroethanone from Step 1 (1.7 g, 6.7 mmol) in MeOH (80 mL) was added solid NaBH₄ (0.5 g, 13.2 mmol) and the mixture was stirred for 30 min followed by quenching with AcOH (1 mL). The solvent was removed and the residue was diluted with water (50 mL) and extracted with EtOAc (3×50 mL). The combined organic extracts were washed with sat. aq. NaHCO₃ (1×50 mL), brine (1×50 mL) and dried (Na₂SO₄). Concentration of the dried extracts afforded the title compound as a light brown oil.

¹H NMR δ (ppm)(Acetone): 8.70 (1H, d, J=2.0 Hz), 8.13 (1H, dd, J=2.3, 8.4 Hz), 7.66 (1H, d, J=8.4 Hz), 6.02 (1H, d, J=6.5 Hz), 5.26-5.20 (1H, m).

### Step 3: Synthesis of 1-{4-[6-(2,2,2-trifluoro-1-hydroxyethyl) yridine-3-yl]phenyl}cyclopropanecarboxamide

To a solution 1-(5-bromopyridin-2-yl)-2,2,2-trifluoroethanol from Step 2 (128 mg, 0.5 mmol), 1-[4-(4,4,5,5-tetramethyl-1,3,2-dioxaborolan-2-yl)phenyl]cyclopropanecarboxamide from Step 5, Example 3 (220 mg, 0.77 mmol) and PdCl₂(dppf) (41 mg, 0.05 mmol) in dry DMF (5 mL) was added aqueous Na₂CO₃ (2 M, 900 μL, 1.8 mmol). The solution was heated at 85° C. for 3 h. Et₂O (50 mL) and water (50 mL) were added and the layers were separated. The aqueous phase was extracted with Et₂O (3×50 mL) and the combined organic extracts were washed with brine (1×50 mL), dried (Na₂SO₄) and concentrated. The resulting residue was flash chromatographed (5:95 EtOH/CH₂Cl₂) to afford the title compound as a white powder.

¹H NMR δ (ppm)(Acetone): 8.90 (1H, d, J=1.9 Hz), 8.19 (1H, dd, J=2.3, 8.2 Hz), 7.76-7.74 (3H, m), 7.59 (2H, d, J=8.3 Hz), 6.37 (1H, s), 5.92 (1H, d, J=6.6 Hz), 5.88 (1H, s), 5.31-5.25 (1H, m), 1.47 (2H, q, J=3.4 Hz), 1.04 (2H, q, J=3.4 Hz). MS (+ESI): 337 (M+1)⁺

### EXAMPLE 8

### Synthesis of (1S)-1-(4-bromophenyl)-2,2,2-trifluoroethanamine

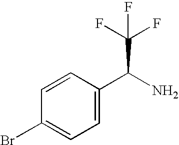

### Step 1: Synthesis of 1-(4-bromophenyl)-2,2,2-trifluoroethanimine

1-(4-Bromophenyl)-2,2,2-trifluoroethanone (491 mg, 2.82 mmol) was dissolved in toluene (10 mL) at rt. A solution of lithium bis(trimethylsilylamide) (1 M in THF, 3.15 mL, 3.15 mmol) was added over a 10 min period. The reaction was stirred at rt for 15 min and then concentrated to yield N-[1-(4-bromophenyl)-2,2,2-trifluoroethylidene]-1,1,1-trimethylsilanamine. Solvolysis of the N-TMS-ketimine took place by stirring at rt for 16 h in MeOH. Upon removal of MeOH, the title compound was generated.

### Step 2: Synthesis of (1S)-1-(4-bromophenyl)-2,2,2-trifluoroethanamine

A solution of (R)-B-butyl-diphenylpyrrolidino-oxazaborolidine (0.3 M in toluene, 3.14 mL, 0.94 mmol) was dissolved in toluene (10 mL) and cooled to −15° C., and catecholborane (6.01 mL, 56.5 mmol) was added to the solution. A solution of 1-(4-bromophenyl)-2,2,2-trifluoroethanamine from Step 1 (10 g, 37.6 mmol) in toluene (40 mL) was added dropwise via syringe pump over a period of 2.5 h. After the addition was complete, the reaction mixture was stirred at −15° C. for 18 h. The reaction was quenched with aqueous 1 N HCl (50 mL) and allowed to warm to room temperature, and the layers were separated. The aqueous layer was basified with 10 N NaOH to Ph 12. The aqueous layer was extracted with MTBE (1×50 mL). The layers were separated, and the organic layer was washed with aqueous 2 N NaOH (2×50 mL) and water (50 mL). The organic layer was treated with Amberlite IRC-50S ion-exchange resin (5 g) for 40 min to remove ®-diphenylprolinol and filtered. The organic layer was dried and filtered. A solution of hydrogen chloride (2 M in Et₂O, 40 mL) was added to the crude solution of amine. A white precipitate formed. After aging at rt for 1 h, the slurry was filtered and the solids were washed with MTBE (10 mL) to afford (1S)-1-(4-bromophenyl)-2,2,2-trifluoroethanamine hydrochloride as a white powder.

¹H NMR δ (ppm)(CD₃OD): 7.73 (2H, d, J=8.5), 7.51 (2H, d, J=8.5),5.42 (1H, q, J=7.4).

### EXAMPLE 9

### Synthesis of (1R)-1-(4-bromophenyl)-2,2,2-trifluoroethanamine

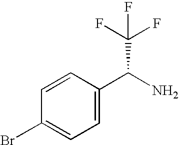

The title compound was synthesized in an identical manner to (1S)-1-(4-bromophenyl)-2,2,2-trifluoroethanamine (Example 8) using (S)-B-butyl-diphenylpyrrolidino-oxazaborolidine as catalyst.

### EXAMPLE 10

### Synthesis of [1-(4-bromophenyl)-2,2,2-trifluoroethyl]amine

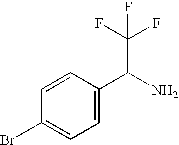

1-(4-Bromophenyl)-2,2,2-trifluoroethanone (491 mg, 2.82 mmol) was dissolved in toluene (10 mL) at rt. A solution of lithium bis(trimethylsilylamide) (1 M in THF, 3.15 mL, 3.15 mmol) was added over a 10 min period. The reaction was stirred at rt for 15 min. and BH₃.Me₂S (2M in toluene, 2.82 mL, 5.73 mmol) was added. The reaction mixture was stirred at rt for 20 min. After cooling to 0° C., aq. 2 N NaOH (4 mL) was carefully added dropwise over 5 min. The mixture was stirred at rt for 90 min. The layers were separated, and the organic layer was washed with aqueous 2 N NaOH (5 mL) and water (5 mL), dried (MgSO₄), and filtered. Concentration of the filtrate provided the title compound. Alternatively, to the solution of crude free amine in toluene was added a solution of HCl (4 M in 1,4-dioxane, 1 mL). A white precipitate formed and the solids were washed with MTBE (10 mL) to afford [1-(4-bromophenyl)-2,2,2-trifluoroethyl]amine hydrochloride as a white powder.

### EXAMPLE 11

### Synthesis of [1-(4-bromophenyl)-2,2-difluoroethyl]amine

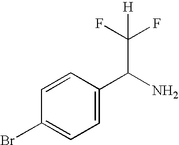

The same procedure used to synthesize of [1-(4-bromophenyl)-2,2,2-trifluoroethyl]amine (Example 10) was used to generate the title compound. The only difference is that 1-(4-bromophenyl)-2,2-difluoroethanone was used as the starting material.

### EXAMPLE 12

### Synthesis of 1-[2-fluoro-4′-(1-hydroxy-1-methylethyl)biphenyl-4-yl]cyclopropanecarbonitrile

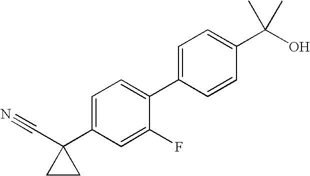

### Step 1: Synthesis of 2-(4-bromophenyl)propan-2-ol

To a cold (0° C.) solution of 1-(4-bromophenyl)ethanone (10 g, 50.2 mmol) in benzene (50 mL) was added MeMgBr (3M in Et₂O, 22.3 mL, 66.8 mmol). The reaction was stirred at 0° C. for 30 minutes and then quenched with aq. NH₄Cl. The mixture was extracted with 40% EtOAc/hexanes, dried (NaSO₄) and concentrated under vacuum. The residue was purified by chromatography on silica gel (EtOAc/Toluene, 0:1 to 1:9) to afford the title compound as light beige solid powder.

¹H NMR δ (ppm)(Acetone): 7.5 (2H, d), 7.4 (2H, d), 1.6 (6H, s).

### Step 2: Synthesis of 2-[4-(4,4,5,5-tetramethyl-1,3,2-dioxaborolan-2-yl)phenyl]propan-2-ol

A solution of 2-(4-bromophenyl)propan-2-ol (6 g, 28 mmol), potassium acetate (8.2 g, 84 mmol) and bis(pinacolato)diboron (10.6 g, 42 mmol) in DMF (80 mL) was degassed followed by the addition of PdCl₂dppf (1.14 g, 1.4 mmol). The reaction mixture was stirred at 90° C. overnight and then diluted with water and extracted with EtOAc (3×). The combined organic extracts were washed with brine (1×), dried (NaSO₄) and concentrated under reduced pressure. The residue was purified by chromatography on silica gel (EtOAc/Toluene, 0:1 to 1:9) and then swished with hexanes overnight to afford, after filtration, the title compound as light beige solid powder.

¹H NMR δ (ppm)(Acetone): 7.71 (2H, d), 7.55 (2H, d), 4.10 (1H, s), 1.58 (6H, s), 1.38 (12H, s).

### Step 3: Synthesis of 1-[2-fluoro-4′-(1-hydroxy-1-methylethyl)biphenyl-4-yl]cyclopropanecarbonitrile

A solution of 2-[4-(4,4,5,5-tetramethyl-1,3,2-dioxaborolan-2-yl)phenyl]propan-2-ol from step 2 above (1.35 g, 5.2 mmol) and 1-(4-bromo-3-fluorophenyl)cyclopropanecarbonitrile from step 6, Example 1 (1.03 g, 4.3 mmol) in DMF (21 mL) was degassed followed by the addition of PdCl₂dppf (350 mg, 0.4 mmol) and 2M aqueous Na₂CO₃ (6.4 mL, 12.9 mmol). The reaction mixture was stirred at 90° C. for 4 h and then diluted with water and extracted with EtOAc (4×). The combined organic extracts were washed with brine (2×), dried (NaSO₄) and concentrated under reduced pressure. The residue was purified by chromatography on silica gel (EtOAc/Hexanes, 0:1 to 2:8 to 3:7) to afford the title compound as white solid powder.

¹H NMR δ (ppm)(Acetone): 7.67 (2H, d, J=8.4 Hz), 7.60 (1H, d, J=8.1 Hz), 7.57-7.53 (2H, m), 7.35 (1H, dd, J=2.0, 8.0 Hz), 7.22 (1H, dd, J=2.0, 12.1 Hz), 4.13 (1H, s), 1.85 (2H, q, J=4.2 Hz), 1.67-1.63 (2H, m), 1.58 (6H, s).

### EXAMPLE 13

### Synthesis of 1-(2-fluoro-1,1′:4′,1″-terphenyl-4-yl)cyclopropanecarbonitrile

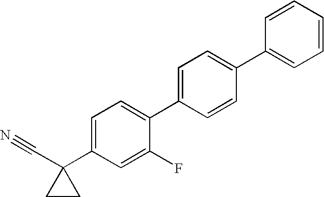

### Step 1: Synthesis of 1-[3-fluoro-4-(4,4,5,5-tetramethyl-1,3,2-dioxaborolan-2-yl)phenyl]cyclopropanecarbonitrile

A solution of 1-(4-bromo-3-fluorophenyl)cyclopropanecarbonitrile from Step 6, Example 1 (2 g, 8.3 mmol), potassium acetate (2.4 g, 24.4 mmol) and bis(pinacolato)diboron (3.2 g, 12.5 mmol) in DMF (25 mL) was degassed followed by the addition of PdCl₂dppf (0.34 g, 0.4 mmol). The reaction mixture was stirred at 85° C. overnight and then diluted with water and extracted with EtOAc (3×). The combined organic extracts were washed with brine (1×), dried (NaSO₄) and concentrated under reduced pressure. The residue was swished with hexanes overnight to afford, after filtration, the title compound as light beige solid powder.

¹H NMR δ(ppm)(Acetone): 7.74 (1H, t, J=7.0 Hz), 7.26 (1H, d, J=6.6 Hz), 7.06 (1H, d, J=10.3 Hz), 1.85 (2H, q, J=4.4 Hz), 1.66-1.62 (2H, m), 1.37 (12H, s).

### Step 2: Synthesis of 1-(2-fluoro-1.1′:4′,1″-terphenyl-4-yl)cyclopropanecarbonitrile

A solution of 1-[3-fluoro-4-(4,4,5,5-tetramethyl-1,3,2-dioxaborolan-2-yl)phenyl]cyclopropanecarbonitrile from Step 1 above (296 mg, 1.0 mmol) and 4-bromobiphenyl (200 mg, 0.86 mmol) in DMF (5 mL) was degassed followed by the addition of PdCl₂dppf (35 mg, 0.04 mmol) and 2M aqueous Na₂CO₃ (1.3 mL, 2.6 mmol). The reaction mixture was stirred at 90° C. overnight and then diluted with water and extracted with EtOAc (3×). The combined organic extracts were washed with brine (1×), dried (NaSO₄) and concentrated under reduced pressure. The residue was purified by chromatography on silica gel (EtOAc/Toluene, 0:1 to 1:9) and then swished with hexanes overnight to afford, after filtration, the title compound as white solid powder.

¹HNMR δ (ppm)(Acetone): 8.31 (2H, d, J=8.3 Hz), 8.26-8.20 (4H, m), 8.15 (1H, t, J=8.1 Hz), 8.02 (2H, t, J=7.7 Hz), 7.93 (1H, d, J=7.5 Hz), 7.88 (1H, dd, J=2.0, 8.2 Hz), 7.75 (1H, dd, J=2.0, 12.1 Hz), 2.36 (2H, q, J=4.3 Hz), 2.19-2.15 (2H, m).

Using similar experimental procedures as those listed above, the following compounds were synthesized.

| EX. | NAME | CHARACTERIZATION |
|---|---|---|
| 14 | 1-[4′-(1-amino-2,2- | MS (+APCI): 317.1 (M + 1)+, 300.1 (M − NH2]⁺ |
| | difluoroethyl)biphenyl-4- | |
| | yl]cyclopropanecarboxamide | |
| 15 | 1-{4′-[(1S)-1-amino-2,2- | MS (+ESI): 357.1 [M + 1]⁺ |
| | difluoroethyl]biphenyl-4-yl}-N- | |
| | cyclopropylcyclopropanecarboxamide | |
| 16 | 1-{4′-[(1S)-1-amino-2,2,2-trifluoroethyl]- | MS (+ESI): 353.0 [M + 1]⁺ |
| | 2-fluorobiphenyl-4- | |
| | yl}cyclopropanecarboxamide | |
| 17 | 1-{2-fluoro-4′-[(1R)-2,2,2-trifluoro-1- | MS (+ESI): 354.2 [M + 1]⁺ |
| | hydroxyethyl]biphenyl-4- | |
| | yl}cyclopropanecarboxamide | |
| 18 | 1-{4′-[(1S)-2,2-difluoro-1- | MS (+ESI): 318.0 [M + 1]⁺ |
| | hydroxyethyl]biphenyl-4- | |
| | yl}cyclopropanecarboxamide | |
| 19 | 1-{4′-[(1R)-2,2-difluoro-1- | MS (+ESI): 318.0 [M + 1]⁺ |
| | hydroxyethyl]biphenyl-4- | |
| | yl}cyclopropanecarboxamide | |
| 20 | 1-[4′-(1-amino-2,2-difluoroethyl)-2- | MS (+ESI): 335.2 [M + 1]⁺ |
| | fluorobiphenyl-4- | |
| | yl]cyclopropanecarboxamide | |
| 21 | 2-{4′-[(1R)-2,2,2-trifluoro-1- | MS (−ESI): 323.2 [M − 1]⁻ |
| | hydroxyethyl]biphenyl-4-yl}propanoic | |
| | acid | |
| 22 | (2S)-2-{4′-[(1R)-2,2,2-trifluoro-1- | MS (−ESI): 323.2 [M − 1]⁻ |
| | hydroxyethyl]biphenyl-4-yl}propanoic | |
| | acid | |
| 23 | (2S)-2-{4′-[(1R)-2,2,2-trifluoro-1- | MS (+ESI): 324.1 [M + 1]⁺ |
| | hydroxyethyl]biphenyl-4-yl}propanamide | |
| 24 | 1-[4′-(1-amino-2,2- | MS (+ESI): 301.0 [M + 1]⁺ |
| | difluoroethyl)biphenyl-4- | |
| | yl]cyclopropanecarboxylic acid | |
| 25 | 2-[4′-(2,2-difluoro-1-hydroxyethyl)-2- | MS (+ESI): 310.0 [M + 1]⁺ |
| | fluorobiphenyl-4-yl]acetamide | |
| 26 | 2,2-difluoro-1-[4-(4-methyl-1,3-thiazol-2- | MS (+ESI): 256.0 [M + 1]⁺ |
| | yl)phenyl]ethanol | |
| 27 | 1-[4′-(2,2-difluoro-1- | MS (+ESI): 307.0 [M + 1]⁺ |
| | hydroxyethyl)biphenyl-4-yl]-2- | |
| | methylpropan-2-ol | |
| 28 | 1-{6-[4′-(2,2-difluoro-1- | MS (+ESI): 292.0 [M + 1]⁺ |
| | hydroxyethyl)phenyl]pyridin-3- | |
| | yl}cyclopropanol | |
| 29 | 1-[4′-(2,2-difluoro-1-hydroxyethyl)-2- | 1H NMR δ (ppm)(Acetone): 7.72 (1H, d, J = 8.3 Hz), |
| | fluorobiphenyl-4-yl]cyclopropanol | 7.60 (3H, m), 7.52-7.45 (1H, m), |
| | | 7.24-7.16 (2H, m), 6.00-5.94 (1H, m), 5.34 (1H, dd, J = 3.3, |
| | | 4.9 Hz), 5.17 (1H, s), 4.97-4.90 (1H, m), |
| | | 1.27-1.20 (2H, m), 1.12-1.10 (2H, m). |
| 30 | (1R)-1-{4′-[(1S)-1-amino-2,2,2- | MS (+ESI): 315.0 [M + 1]⁺ |
| | trifluoroethyl]biphenyl-4-yl}-2,2- | |
| | difluoroethanol | |
| 31 | 2-[4′-(2,2-difluoro-1-hydroxyethyl)-2- | MS (+ESI): 338.0 [M + 1]⁺ |
| | fluorobiphenyl-4-yl]-2- | |
| | methylpropanamide | |
| 32 | 2-[4′-(1-amino-2,2-difluoroethyl)-2- | MS (+ESI): 320.0 [M − NH₂]+ |
| | fluorobiphenyl-4-yl]-2- | |
| | methylpropanamide | |
| 33 | 2-{2-fluoro-4′-[(1R)-2,2,2-trifluoro-1- | MS (+ESI): 356.1 [M + 1]⁺ |
| | hydroxyethyl]biphenyl-4-yl}-2- | |
| | methylpropanamide | |
| 34 | 1-[4′-(2,2-difluoro-1- | MS (+ESI): 318 [M + 1]+ |
| | hydroxyethyl)biphenyl-4- | |
| | yl]cyclopropanecarboxamide | |
| 35 | 1-{4-[6-(2,2,2-trifluoro-1- | MS (+ESI): 337 [M + 1]⁺ |
| | hydroxyethyl)pyridin-3- | |
| | yl]phenyl}cyclopropanecarboxamide | |
| 36 | 1-{3-fluoro-4-[6-(2,2,2-trifluoro-1- | MS (+ESI): 355 [M + 1]⁺ |
| | hydroxyethyl)pyridin-3- | |
| | yl]phenyl}cyclopropanecarboxamide | |
| 37 | 1-biphenyl-4-yl-2,2,2-trifluoroethanol | ¹H NMR (acetone-d₆) δ 7.70 (6H, m), |
| | | 7.50-7.30 (3H, m), 5.90 (1H, d), 5.28 (1H, m). |
| 38 | (1-biphenyl-4-yl-2,2,2- | MS (+ESI): 252 [M + 1]⁺ |
| | trifluoroethyl)amine | |
| 39 | 2,2-difluoro-1-{4′-[(1R)-2,2,2-trifluoro-1- | ¹H NMR (acetone-d₆) δ 8.20 (2H, d), 7.96 (2H, |
| | hydroxyethyl]biphenyl-4-yl}ethanone | d), 7.82 (2H, d), 7.70 (2H, d), 6.98 (1H, t), |
| | | 6.00 (1H, d), 5.32 (1H, m). |
| 40 | 1-{4′-[(1S)-1-amino-2,2,2- | MS (+ESI): 313 [M − NH₂]⁺ |
| | trifluoroethyl]biphenyl-4-yl}-2,2- | |
| | difluoroethanone | |
| 41 | 1,1-difluoro-2-{4′-[(1R)-2,2,2-trifluoro-1- | ¹H NMR (acetone-d₆) δ 7.70 (8H, m), 5.92 (2H, |
| | hydroxyethyl]biphenyl-4-yl}propan-2-ol | m), 5.28 (1H, m), 5.04 (1H, s), 1.66 (3H, s). |
| 42 | 2-{4′-[(1S)-1-amino-2,2,2- | MS (+ESI): 346 [M + 1]⁺ |
| | trifluoroethyl]biphenyl-4-yl}-1,1- | |
| | difluoropropan-2-ol | |
| 43 | 1-[4′-(2,2-difluoro-1-hydroxyethyl)-2- | see Example 1 |
| | fluorobiphenyl-4- | |
| | yl]cyclopropanecarbonitrile | |
| 44 | 1-[4′-(2,2-difluoro-1-hydroxyethyl)-2- | MS (+ESI): 336.0 [M + 1]⁺ |
| | fluorobiphenyl-4- | |
| | yl]cyclopropanecarboxamide | |
| 45 | 2,2-difluoro-1-[4′- | MS (+ESI): 313.0 [M + 1]⁺ |
| | (methylsulfonyl)biphenyl-4-yl]ethanol | |
| 46 | 2,2,2-trifluoro-1-[4′- | 1H NMR δ (ppm)(Acetone): 8.04 (2H, d, J = 8.5 Hz), |
| | (methylsulfonyl)biphenyl-4-yl]ethane-1,1- | 7.96 (2H, t, J = 4.3 Hz), 7.88-7.78 (4H, |
| | diol | m), 6.67 (2H, s), 3.17 (3H, s). |
| 47 | N-cyclopropyl-1-[4′-(2,2-difluoro-1- | MS (+ESI): 376.3 [M + 1]⁺ |
| | hydroxyethyl)-2-fluorobiphenyl-4- | |
| | yl]cyclopropanecarboxamide | |
| 48 | 1-[4′-(1-amino-2,2-difluoroethyl)-2- | MS (+ESI): 358.2 [M − NH₂]⁺ |
| | fluorobiphenyl-4-yl]-N- | |
| | cyclopropylcyclopropanecarboxamide | |
| 49 | 1-{4′-[(1R)-1-amino-2,2,2-trifluoro-1- | MS (+ESI): 332.1 [M − NH₂]⁺ |
| | methylethyl]biphenyl-4- | |
| | yl}cyclopropanecarboxamide | |
| 50 | 1-[4′-(2,2,2-trifluoro-1-hydroxy-1- | MS (+ESI): 350.1 [M + 1]⁺ |
| | methylethyl)biphenyl-4- | |
| | yl]cyclopropanecarboxamide | |
| 51 | 1-{4′-[(2,4- | MS (+ESI): 380.1 [M + 1]⁺ |
| | difluorophenyl)(hydroxy)methyl]biphenyl- | |
| | 4-yl}cyclopropanecarboxamide | |
| 52 | 1-{4′-[amino(2,4- | MS (+ESI): 378.0 [M + 1]⁺ |
| | difluorophenyl)methyl]biphenyl-4- | |
| | yl}cyclopropanecarboxamide | |
| 53 | 1-[4′-(2,2-difluoro-1-hydroxyethyl)-3′- | |
| | fluorobiphenyl-4- | |
| | yl]cyclopropanecarboxamide | |
| 54 | (1R)-1-[4′-(2,2-difluoro-1- | MS (−ESI): 330.1 [M − 1]⁻ |
| | hydroxyethyl)biphenyl-4-yl]-2,2,2- | |
| | trifluoroethanol | |
| 55 | 1-{4′-[(1S)-1-amino-2,2,2- | MS (+ESI): 332.2 [M + 1]⁺ |
| | trifluoroethyl]biphenyl-4-yl}-2,2- | |
| | difluoroethanol | |
| 56 | {(1S)-2,2,2-trifluoro-1-[4′- | MS (+APCI): 330.0 (M + 1)+, 313.0 [M − NH2]⁺ |
| | (methylsulfonyl)biphenyl-4- | |
| | yl]ethyl}amine | |
| 57 | 1-{4′-[(1S)-1-amino-2,2,2- | MS (+APCI): 336.0 (M + 1)+, 319.0 [M − NH2]⁺ |
| | trifluoroethyl]biphenyl-4- | |
| | yl}cyclopropanecarboxylic acid | |
| 58 | 1-{4′-[(1S)-1-amino-2,2- | MS (+APCI): 317.1 (M + 1)+, 300.1 (M − NH2]⁺ |
| | difluoroethyl]biphenyl-4- | |
| | yl}cyclopropanecarboxamide | |
| 59 | 2-[4′-(1-amino-2,2,2- | MS (+APCI): 323.2 (M + 1)+, 306.2 [M − NH2]⁺ |
| | trifluoroethyl)biphenyl-4-yl]propanamide | |
| 60 | 2-{4′-[(1R)-2,2,2-trifluoro-1- | MS (+APCI): 324.2 (M + 1)+ |
| | hydroxyethyl]biphenyl-4-yl}propanamide | |
| 61 | (2S)-2-{2-fluoro-4′-[(1R)-2,2,2-trifluoro- | MS (+APCI): 342.0 (M + 1)+ |
| | 1-hydroxyethyl]biphenyl-4- | |
| | yl}propanamide | |
| 62 | (2S)-2-{4′-[(1S)-1-amino-2,2,2- | MS (+APCI): 341.0 (M + 1)+, 324.0 [M − NH2]⁺ |
| | trifluoroethyl]-2-fluorobiphenyl-4- | |
| | yl}propanamide | |
| 63 | 1-biphenyl-4-yl-2,2,2-trifluoroethanol | ¹H NMR (acetone-d₆) δ 7.70 (6H, m), |
| | | 7.50-7.30 (3H, m), 5.90 (1H, d), 5.28 (1H, m) |
| 64 | (1-biphen-4-yl-2,2,2-trifluoroethyl)amine | MS (+ESI): 252 [M + 1]⁺ |
| 65 | (1R)-1-(4′-bromobiphenyl-4-yl)-2,2- | |
| | difluoroethanol | |
| 66 | 1-{4′-[(1R)-2,2-difluoro-1- | |
| | hydroxylethyl]biphenyl-4-yl}-2,2,2- | |
| | trifluororethanone | |
| 67 | 1-[2-fluoro-4′-(1-hydroxy-1- | ¹H NMR δ (ppm)(Acetone): 7.67 (2H, d, J = 8.4 Hz), |
| | methylethyl)biphenyl-4- | 7.60 (1H, d, J = 8.1 Hz), 7.57-7.53 (2H, m), |
| | yl]cyclopropanecarbonitrile | 7.35 (1H, dd, J = 2.0, 8.0 Hz), 7.22 (1H, dd, J = 2.0, |
| | | 12.1 Hz), 4.13 (1H, s), 1.85 (2H, q, J = 4.2 Hz), |
| | | 1.67-1.63 (2H, m), 1.58 (6H, s). |
| 68 | 1-[2-fluoro-4′-(2,2,2-trifluoro-1- | ¹H NMR δ (ppm)(Acetone): 7.71-7.65 (5H, m), |
| | hydroxylethyl)biphenyl-4- | 7.38 (1H, d, J = 8.2 Hz), 7.25 (1H, d, J = 11.6 Hz), |
| | yl]cyclopropanecarbonitrile | 5.97 (1H, d, J = 5.5 Hz), 5.33 (1H, d, J = 6.4 Hz), |
| | | 1.86 (2H, s), 1.67 (2H, s). |
| 69 | 1-[2-fluoro-4′- | ¹H NMR δ (ppm)(Acetone): 8.24 (2H, d, J = 7.9 Hz), |
| | (trifluoroacetyl)biphenyl-4- | 7.93 (2H, dd, J = 1.7, 8.5 Hz), 7.73 (1H, t, |
| | yl]cyclopropanecarbonitrile | J = 8.2 Hz), 7.44 (1H, dd, J = 2.0, 8.1 Hz), |
| | | 7.32-7.26 (1H, m), 1.91-1.87 (2H, m), 1.70 (2H, q, J = 4.4 Hz). |
| 70 | 1-{2-fluoro-4′-[2,2,2-trifluoro-1- | ¹H NMR δ (ppm)(Acetone): 7.93 (2H, d, J = 8.2 Hz), |
| | hydroxy-1- | 7.78 (2H, d, J = 7.6 Hz), 7.66 (1H, t, J = 8.2 Hz), |
| | (trifluoromethyl)ethyl]biphenyl-4- | 7.40 (1H, d, J = 8.0 Hz), 7.27 (1H, d, J = 12.3 Hz), |
| | yl}cyclopropanecarbonitrile | 4.08 (1H, q, J = 7.2 Hz), 1.87 (2H, q, J = 4.3 Hz), |
| | | 1.70-1.66 (2H, m). |
| 71 | 1-[4′-(2,2,2-trifluoro-1- | ¹H NMR δ (ppm)(Acetone): 7.76-7.66 (6H, m), |
| | hydroxyethyl)biphenyl-4- | 7.50 (2H, d, J = 8.4 Hz), 5.95 (1H, d, J = 5.5 Hz), |
| | yl]cyclopropanecarbonitrile | 5.35-5.29 (1H, m), 1.80 (2H, q, J = 4.2 Hz), |
| | | 1.65-1.57 (2H, m). |
| 72 | 1-[4′-(2,2,2-trifluoro-1- | ¹H NMR δ (ppm)(Acetone): 7.76 (2H, d, J = 8.3 Hz), |
| | hydroxyethyl)biphenyl-3- | 7.67 (4H, dd, J = 8.3, 20.9 Hz), 7.54 (1H, t, |
| | yl]cyclopropanecarbonitrile | J = 7.6 Hz), 7.46 (1H, d, J = 7.8 Hz), 5.95 (1H, |
| | | d, J = 5.5 Hz), 5.32 (1H, t, J = 6.1 Hz), |
| | | 1.83-1.79 (2H, m), 1.69-1.65 (2H, m). |
| 73 | 1-(2-fluoro-4′-isopropylbiphenyl-4- | ¹H NMR δ (ppm)(Acetone): 7.57 (1H, t, J = 8.2 Hz), |
| | yl)cyclopropanecarbonitrile | 7.52 (2H, d, J = 6.8 Hz), 7.39 (2H, d, J = 8.2 Hz), |
| | | 7.35 (1H, d, J = 8.2 Hz), 7.22 (1H, d, J = 12.1 Hz), |
| | | 3.00 (1H, t, J = 7.1 Hz), 1.84 (2H, d, |
| | | J = 2.8 Hz), 1.66 (2H, t, J = 3.7 Hz), 1.30 (6H, d, |
| | | J = 6.9 Hz). |
| 74 | 1-[2-fluoro-4′-(2-hydroxypiperidin-2- | ¹H NMR δ (ppm)(Acetone): 8.09 (1H, s), 7.70 (2H, |
| | yl)biphenyl-4- | d, J = 8.4 Hz), 7.59 (4H, t, J = 8.2 Hz), 7.35 (2H, |
| | yl]cyclopropanecarbonitrile | dd, J = 1.9, 8.1 Hz), 7.22 (1H, dd, J = 1.9, 12.0 Hz), |
| | | 4.38 (1H, s), 4.31 (1H, dd, J = 4.6, 12.6 Hz), |
| | | 3.71-3.59 (2H, m), 3.17-3.11 (1H, m), |
| | | 1.86-1.82 (2H, m), 1.67-1.63 (2H, m). |
| 75 | 1-[2-fluoro-4′-(1- | ¹H NMR δ (ppm)(CDCl3): 7.63-7.57 (4H, m), |
| | hydroxycyclobutyl)biphenyl-4- | 7.47 (1H, t, J = 8.1 Hz), 7.21 (1H, dd, J = 2.0, |
| | yl]cyclopropanecarbonitrile | 8.1 Hz), 7.10 (1H, dd, J = 2.0, 11.5 Hz), |
| | | 2.68-2.62 (2H, m), 2.47-2.41 (2H, m), 2.13-2.05 (2H, |
| | | m), 1.83 (2H, q, J = 4.3 Hz), 1.49 (2H, q, J = 4.4 Hz). |
| 76 | 2,2,2-trifluoro-1-(4′- | ¹H NMR δ (ppm)(Acetone): 7.72 (2H, t, J = 4.2 Hz), |
| | isopropylbiphenyl-4-yl)ethanol | 7.69-7.63 (4H, m), 7.39 (2H, d, J = 8.2 Hz), |
| | | 5.90 (1H, t, J = 4.5 Hz), 5.31-5.25 (1H, m), |
| | | 2.99 (1H, t, J = 6.9 Hz), 1.30 (6H, d, J = 6.9 Hz). |
| 77 | 1-[2-fluoro-4′-(2-hydroxy-2- | ¹H NMR δ (ppm)(DMSO): 1.08 (6H, s), |
| | methylpropyl)biphenyl-4- | 1.60-1.60 (2H, m), 1.80-1.80 (2H, m), 2.68 (2H, s), |
| | yl]cyclopropanecarbonitrile | 4.35 (1H, s), 7.20 (1H, d, J = 12.13 Hz), 7.30 (3H, |
| | | d, J = 7.94 Hz), 7.42 (2H, d, J = 7.77 Hz), |
| | | 7.54 (1H, t, J = 8.25 Hz). |
| 78 | 1-[2-fluoro-3′-(2-hydroxy-2- | ¹H NMR δ (ppm)(DMSO): 1.07 (6H, s), |
| | methylpropyl)biphenyl-4- | 1.60-1.60 (2H, m), 1.80-1.80 (2H, m), 2.70 (2H, s), |
| | yl]cyclopropanecarbonitrile | 4.33 (1H, s), 7.21 (2H, d, J = 14.79 Hz), 7.31 (1H, |
| | | dd, J = 8.05, 2.04 Hz), 7.35 (3H, d, J = 5.01 Hz), |
| | | 7.52 (1H, t, J = 8.19 Hz). |
| 79 | 1-[4-(1-benzothien-3-yl)-3- | ¹H NMR δ (ppm)(DMSO): 1.65-1.65 (2H, m), |
| | fluorophenyl]cyclopropanecarbonitrile | 1.84-1.84 (2H, m), 7.30 (1H, d, J = 11.42 Hz), |
| | | 7.38 (1H, dd, J = 7.98, 2.00 Hz), 7.43-7.43 (2H, |
| | | m), 7.59-7.59 (2H, m), 7.88 (1H, s), 8.08-8.08 (1H, |
| | | m). |
| 80 | 1-[2-fluoro-3′-(1-hydroxy-1- | ¹H NMR δ (ppm)(DMSO): 1.45 (5H, s), |
| | methylethyl)biphenyl-4- | 1.60-1.60 (2H, m), 1.80-1.80 (2H, m), 5.07 (1H, s), |
| | yl]cyclopropanecarbonitrile | 7.21 (1H, d, J = 12.03 Hz), 7.32-7.32 (2H, m), |
| | | 7.39 (1H, t, J = 7.65 Hz), 7.48 (1H, d, J = 7.83 Hz), |
| | | 7.53 (1H, t, J = 8.17 Hz), 7.61 (1H, s). |
| 81 | 1-{2-fluoro-2′- | ¹H NMR δ (ppm)(DMSO): 1.62-1.62 (2H, m), |
| | [hydroxy(phenyl)methyl]biphenyl-4- | 1.81 (2H, d, J = 3.30 Hz), 5.57 (1H, s), 5.80 (1H, |
| | yl}cyclopropanecarbonitrile | d, J = 4.28 Hz), 7.13 (3H, d, J = 7.03 Hz), 7.17 (3H, |
| | | s), 7.26-7.24 (3H, m), 7.35-7.31 (3H, m). |
| 82 | 1-{2-fluoro-4′-[hydroxy(1,3-thiazol-2- | ¹H NMR δ (ppm)(DMSO): 1.60-1.60 (2H, m), |
| | yl)methyl]biphenyl-4- | 1.80-1.80 (2H, m), 6.01 (1H, d, J = 3.81 Hz), |
| | yl}cyclopropanecarbonitrile | 6.87 (1H, s), 7.21 (1H, d, J = 12.08 Hz), 7.32 (1H, |
| | | d, J = 8.18 Hz), 7.46-7.44 (3H, m), |
| | | 7.51-7.51 (2H, m), 7.62 (1H, d, J = 3.30 Hz), 7.69 (1H, d, |
| | | J = 3.33 Hz). |
| 83 | 1-[2-fluoro-3′-(3-hydroxy-3-methyl-2- | ¹H NMR δ (ppm)(DMSO): 1.24 (6H, s), 1.60 (2H, |
| | oxobutyl)biphenyl-4- | s), 1.80 (2H, s), 4.06 (2H, s), 5.43 (1H, s), |
| | yl]cyclopropanecarbonitrile | 7.21 (2H, d, J = 14.51 Hz), 7.34-7.32 (2H, m), |
| | | 7.38 (1H, s), 7.52 (2H, s). |
| 84 | 1-{3-fluoro-4-[5-(1-hydroxy-1- | ¹H NMR δ (ppm)(DMSO): 1.48 (6H, s), |
| | methylethyl)pyridin-2- | 1.63-1.63 (2H, m), 1.82-1.82 (2H, m), 5.25 (1H, s), |
| | yl]phenyl}cyclopropanecarbonitrile | 7.23 (1H, dd, J = 12.56, 1.96 Hz), 7.35-7.35 (1H, |
| | | m), 7.72 (1H, d, J = 8.30 Hz), 7.95-7.95 (2H, m), |
| | | 8.80 (1H, d, J = 2.36 Hz). |
| 85 | 1-[2-fluoro-4′- | ¹H NMR δ (ppm)(DMSO): 1.60-1.60 (2H, m), |
| | (hydroxymethyl)biphenyl-4- | 1.80-1.80 (2H, m), 4.53 (2H, d, J = 5.43 Hz), |
| | yl]cyclopropanecarbonitrile | 5.24 (1H, t, J = 5.68 Hz), 7.21-7.21 (1H, m), |
| | | 7.31 (1H, dd, J = 8.03, 2.01 Hz), 7.41 (2H, d, J = 7.88 Hz), |
| | | 7.49 (2H, d, J = 7.86 Hz), 7.54 (1H, t, |
| | | J = 8.24 Hz). |
| 86 | 1-[2-fluoro-4′-(3-hydroxy-3- | ¹H NMR δ (ppm)(DMSO): 1.14 (6H, s), |
| | methylbutyl)biphenyl-4- | 1.60-1.60 (2H, m), 1.64-1.64 (2H, m), 1.79-1.79 (2H, |
| | yl]cyclopropanecarbonitrile | m), 2.65-2.63 (2H, m), 7.25 (2H, d, J = 7.82 Hz), |
| | | 7.29 (2H, d, J = 7.77 Hz), 7.43 (1H, d, J = 7.77 Hz), |
| | | 7.53-7.53 (2H, m). |
| 87 | 1-[4-(5-acetyl-2-thienyl)-3- | ¹H NMR δ (ppm)(DMSO): 8.00 (1H, d, J = 3.0 Hz), |
| | fluorophenyl]cyclopropanecarbonitrile | 7.94 (1H, t, J = 8.3 Hz), 7.72 (1H, d, J = 4.0 Hz), |
| | | 7.35 (1H, dd, J = 2.0, 8.2 Hz), 7.31 (1H, t, J = 6.3 Hz), |
| | | 1.86 (2H, q, J = 4.3 Hz), 1.66 (2H, q, |
| | | J = 4.5 Hz). |
| 88 | 1-{3-fluoro-4-[5- | ¹H NMR δ (ppm)(DMSO): 1.66-1.66 (2H, m), |
| | (methylsulfonyl)pyridin-2- | 1.86-1.86 (2H, m), 3.34 (3H, s), 7.30 (1H, d, J = 12.71 Hz), |
| | yl]phenyl}cyclopropanecarbonitrile | 7.41-7.41 (1H, m), 8.04-8.04 (2H, m), |
| | | 8.42 (1H, dd, J = 8.27, 2.72 Hz), 9.18 (1H, d, J = 2.54 Hz). |
| 89 | methyl 4′-(1-cyanocyclopropyl)-2′- | ¹H NMR δ (ppm)(DMSO): 1.63-1.63 (2H, m), |
| | fluorobiphenyl-4-carboxylate | 1.82-1.82 (2H, m), 3.87 (3H, s), 7.26 (1H, d, J = 12.15 Hz), |
| | | 7.36 (1H, d, J = 8.16 Hz), 7.62 (1H, t, |
| | | J = 8.22 Hz), 7.70 (2H, d, J = 8.01 Hz), 8.05 (2H, |
| | | d, J = 8.04 Hz). |
| 90 | 1-(4′-benzoyl-2-fluorobiphenyl-4- | ¹H NMR δ (ppm)(DMSO): 1.64 (2H, s), 1.83 (2H, |
| | yl)cyclopropanecarbonitrile | s), 7.27 (1H, d, J = 12.29 Hz), 7.37 (1H, d, J = 8.50 Hz), |
| | | 7.59-7.57 (2H, m), 7.67-7.65 (2H, |
| | | m), 7.76 (4H, dd, J = 18.50, 7.72 Hz), 7.84 (2H, |
| | | d, J = 7.81 Hz). |
| 91 | 1-(3′-acetyl-2-fluorobiphenyl-4- | ¹H NMR δ (ppm)(DMSO): 1.64-1.61 (2H, m), |
| | yl)cyclopropanecarbonitrile | 1.89-1.77 (2H, m), 2.62 (3H, s), 7.26 (1H, d, J = 12.10 Hz), |
| | | 7.35 (1H, d, J = 8.07 Hz), 7.68-7.59 (2H, |
| | | m), 7.80 (1H, d, J = 8.06 Hz), 7.99 (1H, d, J = 7.73 Hz), |
| | | 8.06 (1H, s). |
| 92 | 1-(3′-ethyl-2-fluorobiphenyl-4- | ¹H NMR δ (ppm)(DMSO): 1.20 (3H, t, J = 7.69 Hz), |
| | yl)cyclopropanecarbonitrile | 1.60 (2H, s), 1.80 (2H, s), 2.66 (2H, q, J = 7.79 Hz), |
| | | 7.21 (1H, d, J = 12.20 Hz), 7.25 (1H, |
| | | d, J = 8.16 Hz), 7.40-7.27 (3H, m), 7.58-7.50 (2H, |
| | | m). |
| 93 | 1-[2-fluoro-4′-(2- | ¹H NMR δ (ppm)(DMSO): 1.60 (2H, s), 1.79 (2H, |
| | hydroxyethyl)biphenyl-4- | s), 2.75 (2H, t, J = 6.97 Hz), 3.63 (2H, q, J = 6.41 Hz), |
| | yl]cyclopropanecarbonitrile | 4.66 (1H, t, J = 5.23 Hz), 7.20 (1H, d, |
| | | J = 12.12 Hz), 7.31 (3H, d, J = 7.59 Hz), 7.43 (2H, |
| | | d, J = 7.72 Hz), 7.52 (1H, t, J = 8.22 Hz). |
| 94 | 1-[2-fluoro-4′-(1- | ¹H NMR δ (ppm)(DMSO): 1.34 (3H, d, J = 6.44 Hz), |
| | hydroxyethyl)biphenyl-4- | 1.62-1.57 (2H, m), 1.82-1.78 (2H, m), |
| | yl]cyclopropanecarbonitrile | 4.78-4.72 (1H, m), 5.20 (1H, d, J = 4.22 Hz), |
| | | 7.24-7.18 (1H, m), 7.31 (1H, dd, J = 8.05, 2.01 Hz), |
| | | 7.43 (2H, d, J = 8.03 Hz), 7.47 (2H, d, J = 7.99 Hz), |
| | | 7.54 (1H, t, J = 8.27 Hz). |
| 95 | 1-[2-fluoro-3′-(2- | ¹H NMR δ (ppm)(DMSO): 1.62-1.57 (2H, m), |
| | hydroxyethyl)biphenyl-4- | 1.82-1.78 (2H, m), 2.77 (2H, t, J = 6.92 Hz), |
| | yl]cyclopropanecarbonitrile | 3.63 (2H, q, J = 6.21 Hz), 4.65 (1H, t, J = 5.20 Hz), |
| | | 7.21 (1H, d, J = 12.18 Hz), 7.25 (1H, d, J = 7.31 Hz), |
| | | 7.31 (1H, d, J = 8.62 Hz), 7.38-7.33 (3H, |
| | | m), 7.53 (1H, t, J = 8.17 Hz). |
| 96 | 1-(2-fluoro-1,1′:3′,1″-terphenyl-4- | ¹H NMR δ (ppm)(DMSO): 1.64-1.59 (2H, m), |
| | yl)cyclopropanecarbonitrile | 1.87-1.76 (2H, m), 7.25 (1H, d, J = 12.05 Hz), |
| | | 7.34 (1H, d, J = 8.20 Hz), 7.39 (1H, d, J = 7.52 Hz), |
| | | 7.48 (2H, t, J = 7.57 Hz), 7.53 (1H, d, J = 8.18 Hz), |
| | | 7.57 (1H, t, J = 7.72 Hz), 7.74-7.63 (4H, |
| | | m), 7.77 (1H, s). |
| 97 | 1-(2-fluoro-1,1′:2′,1″-terphenyl-4- | ¹H NMR δ (ppm)(DMSO): 7.56-7.50 (1H, m), |
| | yl)cyclopropanecarbonitrile | 7.47 (2H, t, J = 7.3 Hz), 7.38 (1H, d, J = 7.2 Hz), |
| | | 7.30-7.24 (4H, m), 7.19 (1H, dd, J = 1.8, 8.0 Hz), |
| | | 7.12 (2H, t, J = 3.9 Hz), 6.98 (1H, dd, J = 1.8, |
| | | 11.2 Hz), 1.79 (2H, q, J = 4.3 Hz), 1.55 (2H, |
| | | q, J = 4.3 Hz). |
| 98 | 1-(2-fluoro-1,1′:4′,1″-terphenyl-4- | ¹HNMR δ (ppm)(Acetone): 8.31 (2H, d, J = 8.3 Hz), |
| | yl)cyclopropanecarbonitrile | 8.26-8.20 (4H, m), 8.15 (1H, t, J = 8.1 Hz), |
| | | 8.02 (2H, t, J = 7.7 Hz), 7.93 (1H, d, J = 7.5 Hz), |
| | | 7.88 (1H, dd, J = 2.0, 8.2 Hz), 7.75 (1H, dd, J = 2.0, |
| | | 12.1 Hz), 2.36 (2H, q, J = 4.3 Hz), |
| | | 2.19-2.15 (2H, m). |
| 99 | 1-(2-fluorobiphenyl-4- | ¹H NMR δ (ppm)(Acetone): 7.59-7.43 (6H, m), |
| | yl)cyclopropanecarbonitrile | 7.36 (1H, t, J = 4.0 Hz), 7.23 (1H, t, J = 6.0 Hz), |
| | | 1.85 (2H, d, J = 2.6 Hz), 1.65 (2H, d, J = 2.5 Hz). |
| 100 | 1-(2-fluoro-3′-methylbiphenyl-4- | ¹H NMR δ (ppm)(Acetone): 7.38 (5H, s), 7.25 (2H, |
| | yl)cyclopropanecarbonitrile | s), 2.42 (3H, s), 1.85 (2H, s), 1.65 (2H, s). |
| 101 | 1-(2-fluoro-2′-methylbiphenyl-4- | ¹H NMR δ (ppm)(Acetone): 7.46 (4H, d, J = 3.6 Hz), |
| | yl)cyclopropanecarbonitrile | 7.40 (1H, d, J = 4.3 Hz), 7.34 (2H, d, J = 7.0 Hz), |
| | | 2.31 (3H, s), 1.97 (2H, d, J = 2.6 Hz), |
| | | 1.78 (2H, d, J = 2.4 Hz). |
| 102 | 1-(4′-ethyl-2-fluorobiphenyl-4- | ¹H NMR δ (ppm)(Acetone): 7.58-7.50 (3H, m), |
| | yl)cyclopropanecarbonitrile | 7.36-7.32 (3H, m), 7.21 (1H, dd, J = 1.8, 12.0 Hz), |
| | | 2.72 (2H, q, J = 7.6 Hz), 1.83 (2H, q, J = 4.2 Hz), |
| | | 1.68-1.62 (2H, m), 1.28 (3H, t, J = 7.6 Hz). |
| 103 | 1-(2-fluoro-2′-isopropylbiphenyl-4- | ¹H NMR δ (ppm)(Acetone): 7.47 (2H, d, J = 17.0 Hz), |
| | yl)cyclopropanecarbonitrile | 7.35 (2H, s), 7.29-7.17 (3H, m), 2.85 (1H, |
| | | m, J = 6.6 Hz), 1.86 (2H, s), 1.68 (2H, s), 1.22 (6H, |
| | | d, J = 41.3 Hz). |
| 104 | 1-(2-fluoro-4′-methylbiphenyl-4- | ¹H NMR δ (ppm)(Acetone): 7.57-7.47 (3H, m), |
| | yl)cyclopropanecarbonitrile | 7.32 (3H, t, J = 6.1 Hz), 7.21 (1H, dd, J = 1.8, |
| | | 12.0 Hz), 2.40 (3H, s), 1.83 (2H, q, J = 4.1 Hz), |
| | | 1.68-1.62 (2H, m). |
| 105 | 1-[3-fluoro-4-(2- | ¹H NMR δ (ppm)(Acetone): 8.14 (1H, s), |
| | naphthyl)phenyl]cyclopropanecarbonitrile | 8.04-7.98 (3H, m), 7.76-7.70 (2H, m), 7.61-7.57 (2H, |
| | | m), 7.43-7.37 (1H, m), 7.30-7.24 (1H, m), |
| | | 1.87 (2H, q, J = 4.2 Hz), 1.70-1.64 (2H, m). |
| 106 | 1-(4′-acetyl-2-fluorobiphenyl-4- | ¹H NMR δ (ppm)(Acetone): 8.14-8.10 (2H, m), |
| | yl)cyclopropanecarbonitrile | 7.76-7.72 (2H, m), 7.65 (1H, dd, J = 8.2, 8.2 Hz), |
| | | 7.42-7.31 (1H, m), 7.28-7.24 (1H, m), |
| | | 2.66 (3H, s), 1.88-1.82 (2H, m), 1.73-1.65 (2H, m). |
| 107 | 1-[3-fluoro-4-(1H-indol-5- | ¹H NMR δ (ppm)(Acetone): 10.39 (1H, s), |
| | yl)phenyl]cyclopropanecarbonitrile | 7.81 (1H, s), 7.61-7.50 (2H, m), 7.43-7.31 (3H, m), |
| | | 7.25-7.19 (1H, m), 6.59-6.57 (1H, m), 1.82 (2H, |
| | | q, J = 4.2 Hz), 1.64-1.60 (2H, m). |
| 108 | 1,1′-(2,2′-difluorobiphenyl-4,4′- | ¹H NMR δ (ppm)(Acetone): 7.55-7.49 (2H, m), |
| | diyl)dicyclopropanecarbonitrile | 7.39 (2H, dd, J = 1.9, 8.0 Hz), 7.27-7.23 (2H, |
| | | m), 1.87 (4H, q, J = 4.2 Hz), 1.70-1.66 (4H, m). |
| 109 | 1-(2-fluoro-4′-pyridin-3-ylbiphenyl-4- | ¹H NMR δ (ppm)(Acetone): 8.97 (1H, d, J = 1.8 Hz), |
| | yl)cyclopropanecarbonitrile | 8.63 (1H, dd, J = 1.5, 4.8 Hz), 8.14-8.10 (1H, |
| | | m), 7.86 (2H, d, J = 8.4 Hz), 7.75 (2H, dd, J = 1.6, |
| | | 8.4 Hz), 7.66 (1H, t, J = 8.2 Hz), 7.53-7.49 (1H, |
| | | m), 7.39 (1H, dd, J = 2.0, 8.1 Hz), 7.26 (1H, |
| | | dd, J = 1.9, 12.0 Hz), 1.86 (2H, q, J = 4.3 Hz), |
| | | 1.72-1.66 (2H, m). |
| 110 | 1-(2-fluoro-4′-isopropylbiphenyl-4- | ¹H NMR δ (ppm)(DMSO): 1.31 (6H, s), |
| | yl)cyclopropanecarbonitrile | 1.60-1.60 (2H, m), 1.79-1.79 (2H, m), 7.21 (1H, d, J = 12.10 Hz), |
| | | 7.30 (1H, d, J = 8.65 Hz), |
| | | 7.48-7.48 (4H, m), 7.54 (1H, t, J = 8.33 Hz). |
| 111 | 1-[4′-(1-amino-1-methylethyl)-2- | ¹H NMR δ (ppm)(Acetone): 7.72-7.70 (2H, m), |
| | fluorobiphenyl-4- | 7.58 (1H, t, J = 8.2 Hz), 7.53 (2H, dd, J = 1.7, |
| | yl]cyclopropanecarbonitrile | 8.4 Hz), 7.39-7.33 (1H, m), 7.22 (1H, dd, J = 2.0, |
| | | 12.0 Hz), 2.59 (2H, s), 1.87-1.81 (2H, m), |
| | | 1.69-1.63 (2H, m), 1.50 (6H, s). |
| 112 | [4′-(1-hydroxy-1- | ¹H NMR δ (ppm)(DMSO): 7.70 (2H, d, J = 8.2 Hz), |
| | methylethyl)biphenyl-4-yl]acetonitrile | 7.59 (4H, q, J = 9.5 Hz), 7.44 (2H, d, J = 8.2 Hz), |
| | | 5.06 (1H, s), 4.09 (2H, s), 1.47 (6H, s). |
| 113 | 4′-(1-hydroxy-1-methylethyl)biphenyl- | ¹H NMR (500 MHz, DMSO): δ 1.45 (s, 6H); |
| | 4-carboxamide | 5.07 (s, 1H); 7.55 (d, J = 10.14 Hz, 2H); 7.64 (d, J = 10.45 Hz, |
| | | 2H); 7.72 (d, J = 9.92 Hz, 2H); |
| | | 7.93 (d, J = 11.05 Hz, 2H). |
| 114 | 4′-(1-hydroxy-1-methylethyl)biphenyl- | ¹H NMR δ (ppm)(DMSO): 7.87 (4H, q, J = 7.7 Hz), |
| | 4-sulfonamide | 7.67 (2H, d, J = 8.0 Hz), 7.59 (2H, d, J = 8.0 Hz), |
| | | 7.38 (2H, s), 5.09 (1H, s), 1.46 (6H, s). |
| 115 | 4′-(1-hydroxy-1-methylethyl)biphenyl- | ¹H NMR δ (ppm)(DMSO): 1.45 (6H, s), 5.10 (1H, |
| | 3-carboxamide | s), 7.39 (1H, s), 7.52 (1H, t, J = 7.70 Hz), |
| | | 7.56 (2H, d, J = 8.04 Hz), 7.64 (2H, d, J = 7.99 Hz), |
| | | 7.79 (1H, d, J = 7.59 Hz), 7.82 (1H, d, J = 7.82 Hz), |
| | | 8.09 (1H, s), 8.12 (1H, s). |
| 116 | 2-[4-(1-benzothien-3- | ¹H NMR δ (ppm)(DMSO): 8.07 (1H, t, J = 4.6 Hz), |
| | yl)phenyl]propan-2-ol | 7.90 (1H, t, J = 4.6 Hz), 7.79 (1H, s), |
| | | 7.62 (2H, d, J = 8.4 Hz), 7.55 (2H, d, J = 8.3 Hz), |
| | | 7.47-7.43 (2H, m), 5.09 (1H, s), 1.49 (6H, s). |
| 117 | 1-[4′-(1-hydroxy-1- | ¹H NMR δ (ppm)(DMSO): 1.45 (6H, s), 2.95 (3H, |
| | methylethyl)biphenyl-3-yl]ethanone | s), 5.09 (1H, s), 7.57 (2H, d, J = 8.18 Hz), |
| | | 7.61 (1H, d, J = 7.83 Hz), 7.65 (2H, d, J = 8.06 Hz), |
| | | 7.91 (2H, t, J = 7.00 Hz), 8.16 (1H, s). |
| 118 | 2-[4-(2-naphthyl)phenyl]propan-2-ol | ¹H NMR δ (ppm)(DMSO): 1.56--7.52-7.50 (2H, |
| | | m), 7.57 (2H, d, J = 8.69 Hz), 7.73 (2H, d, J = 8.04 Hz), |
| | | 7.82 (1H, d, J = 9.17 Hz), 7.91 (1H, d, |
| | | J = 8.07 Hz), 7.98 (2H, d, J = 8.26 Hz), 8.17 (1H, |
| | | s). |
| 119 | 1-[4′-(1-hydroxy-1- | ¹H NMR δ (ppm)(DMSO): 8.05 (2H, d, J = 8.4 Hz), |
| | methylethyl)biphenyl-4-yl]ethanone | 7.84 (2H, t, J = 7.2 Hz), 7.69 (2H, t, J = 9.1 Hz), |
| | | 7.62 (2H, t, J = 10.5 Hz), 5.10 (1H, s), |
| | | 2.63 (3H, s), 1.48 (6H, s). |
| 120 | 2-(1,1′:4′,1″-terphenyl-4-yl)propan-2-ol | ¹H NMR δ (ppm)(DMSO): 1.46 (6H, s), 4.85 (1H, |
| | | s), 7.38 (1H, d, J = 8.50 Hz), 7.48 (2H, t, J = 7.84 Hz), |
| | | 7.56 (2H, d, J = 8.54 Hz), 7.64 (2H, d, |
| | | J = 8.17 Hz), 7.71 (2H, d, J = 8.33 Hz), 7.75 (4H, |
| | | s). |
| 121 | 2-(1,1′:2′,1″-terphenyl-4-yl)propan-2-ol | ¹H NMR δ (ppm)(DMSO): 1.41 (6H, s, J = 33.88 Hz), |
| | | 4.96 (1H, s), 7.02 (2H, d, J = 8.50 Hz), |
| | | 7.11 (2H, s), 7.23 (3H, s), 7.30 (2H, d, J = 9.03 Hz), |
| | | 7.40 (4H, d, J = 25.39 Hz). |
| 122 | 2-(1,1′:3′,1″-terphenyl-4-yl)propan-2-ol | ¹H NMR δ (ppm)(DMSO): 7.89 (1H, s), 7.78 (2H, |
| | | t, J = 4.2 Hz), 7.72-7.64 (4H, m), 7.59-7.48 (5H, |
| | | m), 7.41 (1H, t, J = 7.3 Hz), 5.08 (1H, s), |
| | | 1.48 (6H, s). |
| 123 | 2-[4′-(methylsulfonyl)biphenyl-4- | ¹H NMR δ (ppm)(DMSO): 1.46 (6H, s), |
| | yl]propan-2-ol | 3.24 (3H, s), 5.11 (1H, s), 7.59 (2H, d, J = 8.63 Hz), |
| | | 7.68 (2H, d, J = 8.46 Hz), 7.92 (2H, d, J = 9.74 Hz), |
| | | 7.98 (2H, d, J = 9.04 Hz). |
| 124 | 1-[4′-(1-hydroxy-1- | ¹H NMR δ (ppm)(DMSO): 1.44 (6H, s), 1.60 (2H, |
| | methylethyl)biphenyl-3- | m), 1.77-1.75 (2H, m), 5.07 (1H, s), 7.33 (1H, |
| | yl]cyclopropanecarbonitrile | d, J = 7.82 Hz), 7.46 (1H, d, J = 7.84 Hz), |
| | | 7.48 (1H, s), 7.57-7.55 (5H, m). |
| 125 | 2,2′-biphenyl-4,4′-diyldipropan-2-ol | ¹H NMR δ (ppm)(DMSO): 7.59-7.53 (8H, m), |
| | | 5.04 (2H, s), 1.47 (12H, s). |
| 126 | 2-[3′-(methylsulfonyl)biphenyl-4- | ¹H NMR δ (ppm)(DMSO): 1.46 (6H, s), 3.28 (3H, |
| | yl]propan-2-ol | s), 7.59 (2H, d, J = 8.06 Hz), 7.68 (2H, d, J = 8.05 Hz), |
| | | 7.73 (1H, t, J = 7.78 Hz), 7.88 (1H, d, |
| | | J = 7.91 Hz), 8.01 (1H, d, J = 7.91 Hz), 8.12 (1H, |
| | | s). |
| 127 | 1-[2-fluoro-4′-(1-hydroxy-1- | ¹H NMR δ (ppm)(DMSO): 7.57 (3H, d, J = 8.3 Hz), |
| | methylethyl)biphenyl-4- | 7.46 (3H, dd, J = 0.0, 7.9 Hz), 7.27 (1H, t, J = 6.3 Hz), |
| | yl]cyclopropanecarboxylic acid | 5.07 (1H, t, J = 6.9 Hz), 1.48 (8H, d, J = 6.5 Hz), |
| | | 1.25-1.21 (2H, m). |
| 128 | 2-{4′- | ¹H NMR δ (ppm)(DMSO): 7.70 (2H, d, J = 8.2 Hz), |
| | [(methylsulfonyl)methyl]biphenyl-4- | 7.60 (4H, q, J = 10.9 Hz), 7.50 (2H, d, J = 8.2 Hz), |
| | yl}propan-2-ol | 5.07 (1H, s), 4.54 (2H, s), 2.95 (3H, s), |
| | | 1.47 (6H, s). |
| 129 | 1-[4′-(1-hydroxy-1- | ¹H NMR δ (ppm)(DMSO): 7.63-7.53 (6H, m), |
| | methylethyl)biphenyl-4- | 7.42 (2H, d, J = 8.2 Hz), 7.04 (1H, s), 6.24 (1H, |
| | yl]cyclopropanecarboxamide | s), 5.04 (1H, s), 1.46 (6H, s), 1.35 (2H, q, J = 3.4 Hz), |
| | | 0.99 (2H, q, J = 3.5 Hz). |
| 130 | 1-[2-fluoro-4′-(1-hydroxy-1- | ¹H NMR δ (ppm)(DMSO): 7.56 (3H, dd, J = 8.3, |
| | methylethyl)biphenyl-4- | 8.3 Hz), 7.50 (2H, t, J = 7.8 Hz), 7.30 (2H, t, J = 5.8 Hz), |
| | yl]methanesulfonamide | 6.93 (2H, s), 5.07 (1H, s), 4.35 (2H, s), |
| | | 1.47 (6H, s). |
| 131 | 1-{6-[4-(1-hydroxy-1- | ¹H NMR δ (ppm) (DMSO): 1.45 (6H, s), 1.65 (2H, |
| | methylethyl)phenyl]pyridin-3- | s), 1.89 (2H, s), 7.56 (2H, d, J = 8.11 Hz), |
| | yl}cyclopropanecarbonitrile | 7.79 (1H, d, J = 8.49 Hz), 7.94 (1H, d, J = 8.42 Hz), |
| | | 7.99 (2H, d, J = 8.20 Hz), 8.63 (1H, s). |
| 132 | 1-[4′-(1-hydroxy-1- | ¹H NMR δ (ppm)(DMSO): 1.03 (2H, m), 1.37 (2H, |
| | methylethyl)biphenyl-3- | m), 1.44 (6H, s), 5.06 (1H, s), 6.24 (1H, s), |
| | yl]cyclopropanecarboxamide | 7.01 (1H, s), 7.31 (1H, d, J = 7.74 Hz), 7.41 (1H, |
| | | t, J = 7.73 Hz), 7.58-7.56 (6H, m). |
| 133 | 2-(4′-pyridin-3-ylbiphenyl-4- | ¹H NMR δ (ppm)(DMSO): 1.46 (6H, s), 7.57 (2H, |
| | yl)propan-2-ol | d, J = 8.04 Hz), 7.67 (3H, d, J = 7.94 Hz), |
| | | 7.81 (2H, d, J = 8.09 Hz), 7.86 (2H, d, J = 8.08 Hz), |
| | | 8.34 (1H, d, J = 7.80 Hz), 8.66 (1H, d, J = 4.92 Hz), |
| | | 9.04 (1H, s). |
| 134 | 3-[4-(1-hydroxy-1- | ¹H NMR δ (ppm)(DMSO): 1.49 (6H, s), 7.67 (4H, |
| | methylethyl)phenyl]quinoline-2- | s), 7.83 (1H, t, J = 7.40 Hz), 7.94 (1H, t, J = 7.55 Hz), |
| | carbonitrile | 8.16 (2H, t, J = 7.77 Hz), 8.68 (1H, s). |
| 135 | 1-[4′-(1-hydroxy-1- | ¹H NMR δ (ppm)(DMSO): 7.62 (2H, d, J = 7.9 Hz), |
| | methylethyl)biphenyl-4-yl]-N- | 7.57 (4H, q, J = 8.7 Hz), 7.41 (2H, d, J = 7.9 Hz), |
| | methylcyclopropanecarboxamide | 6.75 (1H, d, J = 3.8 Hz), 5.04 (1H, s), |
| | | 2.55 (3H, s), 1.46 (6H, s), 1.34 (2H, t, J = 3.0 Hz), |
| | | 0.97 (2H, t, J = 3.1 Hz). |
| 136 | [({1-[4′-(1-hydroxy-1- | ¹H NMR δ (ppm)(DMSO): 7.65 (2H, d, J = 8.3 Hz), |
| | methylethyl)biphenyl-4- | 7.60 (2H, d, J = 8.5 Hz), 7.55 (3H, d, J = 8.5 Hz), |
| | yl]cyclopropyl}carbonyl)(methylene)-λ⁵- | 7.43 (2H, d, J = 8.2 Hz), 5.05 (1H, s), |
| | azanyl]acetonitrile | 4.00 (2H, d, J = 5.6 Hz), 1.46 (6H, s), 1.42 (2H, |
| | | q, J = 3.5 Hz), 1.08 (2H, q, J = 3.5 Hz). |
| 137 | 2-(4′-isopropoxybiphenyl-4-yl)propan-2- | ¹H NMR (500 MHz, DMSO): δ 1.28 (d, 6H); |
| | ol | 1.44 (s, 6H); 4.64-4.62 (m, 1H); 5.01 (s, 1H); |
| | | 6.97 (d, J = 8.10 Hz, 2H); 7.50 (s, 4H); 7.55 (s, 2H). |
| 138 | 1-[2-fluoro-4′-(1-hydroxy-1- | MS (+ESI): [M − 18]+ |
| | methylethyl)biphenyl-4- | |
| | yl]cyclopropanecarboxamide | |

### MAO Assay

### Solutions

### 5× Buffer

250 mM sodium phosphate pH 7.4: 38.7 ml Na₂HPO₄ 1M
11.3 ml NaH₂PO₄ 1M
150 ml H₂0
   Dilute to 1× in water (11 ml per plate)

### MAO-A

Recombinant human MAO-A

### Sigma # M7316

### Lot# 024K1057

5 mg/mL, 120 U/mg
Dilute to 20 ug/ml in 1× buffer (2.7 ml per plate)

### MAO-B

Recombinant human MAO-B

### Sigma # M7441

### Lot# 053K0345

5 mg/mL, 40 U/mg
Dilute to 80 ug/ml in 1× buffer (2.7 ml per plate)

### Kynuramine

### Sigma #K3250

### MW: 326

Stock prepared at 20 mM in water (6.5 mg/l ml)

### Store at −20° C.

Dilute to 40 uM in 1× buffer (5.5 mL per plate)

### NaOH

Solution 1N in water (5.5 ml per plate)

### Procedure

To black 96-well plate (Microfluorl Black #7005):
25 ul buffer
1 ul compound or DMSO (final 100, 33, 11, 3.7, 1.2, 0.4, 0.14, 0.05, 0.015, 0.005 uM)
25 ul MAO-A 20 ug/ml or MAO-B 80 ug/ml (final 5 or 20 ug/ml respectively) or buffer for background control
Shake
Incubate at RT for 10 min.
Add 50 uL kynuramine 40 uM (final 20 uM). Shake. Incubate at 37° C. for 30 min for MAO-A and 40 min for MAO-B.
Stop reaction by adding 50 uL of NaOH 1N. Shake.
Read in SpectraMax®Gemini (endpoint mode, top read, Exc=312 nm, Em=425 nm, cutoff 420 nm, 30 read/well, PMT=high); readout should be 1000-2000 FU for DMSO controls.
Calculate IC₅₀ with Softmax® Pro.

1. A compound of the formula: 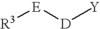
wherein Y is hydrogen, C(R¹)(R²)X, C(O)R¹, C(O)R², C(O)OR¹, CH(OH)R², (C₁₋₆alkyl)C(O)CR¹R²OH, (C₁₋₆alkyl)CR¹R²OH, (C₁₋₆alkyl)OH, SO₂R², C₁₋₆ alkyl, aryl, heteroaryl, C₃₋₈ cycloalkyl or heterocyclyl wherein each said aryl, heteroaryl, cycloalkyl and heterocyclyl groups, which may be monocyclic or bicyclic, is optionally substituted on either the carbon or the heteroatom with one to five substituents independently selected from C₁₋₆ alkyl, halo, cyano or hydroxyl;
X is hydrogen, NH₂ or OH;
R¹ is hydrogen or C₁₋₆ alkyl which is optionally substituted with one to six halo, hydroxyl, O(C₁₋₆ alkyl) or carbonyl;
R² is hydrogen, C₁₋₆ alkyl, aryl, heteroaryl, arylalkyl, heteroarylalkyl or hydroxyl wherein said alkyl, aryl, heteroaryl, haloalkyl, arylalkyl and heteroarylalkyl groups are optionally substituted with one to six halo;
or R¹ and R² can be taken together with the carbon atom to which they are attached to form a C₃₋₈ cycloalkyl ring which is optionally substituted with one to six halo;
D is aryl, heteroaryl, C₃₋₈ cycloalkyl or heterocyclyl wherein each said aryl, heteroaryl, cycloalkyl and heterocyclyl groups, which may be monocyclic or bicyclic, is optionally substituted on either the carbon or the heteroatom with one to five substituents independently selected from the group consisting of C₁₋₆ alkyl, haloalkyl, halo or cyano;
E is aryl, heteroaryl, C₃₋₈ cycloalkyl or heterocyclyl wherein each said aryl, heteroaryl, cycloalkyl and heterocyclyl groups, which may be monocyclic or bicyclic, is optionally substituted on either the carbon or the heteroatom with one to five substituents independently selected from the group consisting of C₁₋₆ alkyl, haloalkyl, halo or cyano;
R³ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR⁵, —C(O)OSi[CH(CH₃)₂]₃, —OR⁴, —OR⁵, —C(O)R⁵, —R⁵C(O)R⁴, —C(O)R⁴, —C(O)N(R^{a})(R^{b}), —C(O)N(R⁷)(R⁷), —C(O)N(R⁵)(R⁶), —C(R^{a})(R^{b})OH, —SR⁷, —SR⁴, —R⁵SR⁴, —R⁴, —C(R⁴)₃, —C(R⁵)(R⁶)N(R⁴)₂, —NR⁵C(O)NR⁵S(O)₂R⁴, —SO₂R⁵, —SO(R⁷), —SO₂R⁴, —SOₘN(R^{c})(R^{d}), —SOₘCH(R⁵)(R⁶), —SO₂N(R⁵)C(O)(R⁷), —SO₂(R⁵)C(O)N(R⁷)₂, —OSO₂R⁵, —N(R⁵)(R⁶), —N(R⁵)C(O)N(R⁵)(R⁴), —N(R⁵)C(O)R⁴, —N(R⁵)C(O)R⁵, —N(R⁵)C(O)OR⁵, —N(R⁵)SO₂(R⁵), —C(R⁵)(R⁶)NR⁵C(R⁵)(R⁶)R⁴, —C(R⁵)(R⁶)N(R⁵)R⁴, —C(R⁵)(R⁶)N(R⁵)(R⁶), —C(R⁵)(R⁶)SC(R⁵)(R⁶)(R⁴), R⁵S—, —C(R^{a})(R^{b})NR^{a}C(R^{a})(R^{b})(R⁴), —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁴, —C(O)C(R^{a})(R^{b})S(R^{a}), C(R^{a})(R^{b})C(O)N(R^{a})(R^{b}), C(R^{a})(R^{b})C(O)OH, —B(OH)₂, —OCH₂O— or 4,4,5,5-tetramethyl-1,3,2-dioxaborolan-2-yl; wherein said alkyl, alkenyl, alkynyl, alkyloxy, aryl, heteroaryl, cycloalkyl and heterocyclyl groups are optionally substituted on either the carbon or the heteroatom with one to five substituents independently selected from C₁₋₆ alkyl, halo, keto, cyano, haloalkyl, hydroxyalkyl, —OR⁴, —NO₂, —NH₂, —NHS(O)₂R⁵, —R⁴SO₂R⁷, —SO₂R⁷, —SO(R⁷), —SR⁷, —SR⁴, —SOₘN(R^{c})(R^{d}), —SOₘN(R⁵)C(O)(R⁷), —C(R⁵)(R⁶)N(R⁵)(R⁶), —C(R⁵)(R⁶)OH, —COOH, —C(R^{a})(R^{b})C(O)N(R^{a})(R^{b}), —C(O)(R^{a})(R^{b}), —C(O)NH₂, —C(O)NHR⁴, —N(R⁵)C(R⁵)(R⁶)(R⁴), —N(R⁵)CO(R⁴), —NH(CH₂)₂OH, —NHC(O)OR⁵, —Si(CH₃)₃, heterocycyl, aryl, or heteroaryl;
R⁴ is hydrogen, aryl, aryl(C₁₋₄) alkyl, heteroaryl, heteroaryl(C₁₋₁₄)alkyl, C₃₋₈cycloalkyl, C₃₋₈cycloalkyl(C₁₋₄)alkyl or heterocyclyl(C 14)alkyl wherein said groups are optionally substituted with one, two, or three substituents independently selected from halo, alkoxy or —SO₂R⁷;
R⁵ is hydrogen or C₁₋₆ alkyl;
R⁶ is hydrogen or C₁₋₆ alkyl;
R⁷ is hydrogen or C₁₋₆ alkyl which is optionally substituted with one, two, or three substituents independently selected from halo, alkoxy, cyano, —NR⁵ or —SR⁵;
R^{a} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl or heterocyclyl, wherein said alkyl, aryl, heteroaryl, cycloalkyl and heterocyclyl groups are optionally substituted on either the carbon or the heteroatom with one, two, or three substituents independently selected from C₁₋₆ alkyl or halo;
R^{b} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl or heterocyclyl, wherein said alkyl, aryl, heteroaryl, cycloalkyl and heterocyclyl groups are optionally substituted on either the carbon or the heteroatom with one, two, or three substituents independently selected from C₁₋₆ alkyl or halo;
or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with one or two substituents independently selected from C₁₋₆ alkyl and halo;
each m is independently selected from an integer from zero to two;
or a pharmaceutically acceptable salt, stereoisomer or N-oxide derivative thereof.

2. The compound of claim 1 wherein D is aryl; E is aryl or heteroaryl, wherein said aryl or heteroaryl group is optionally substituted on either the carbon or the heteroatom with one to five substituents independently selected from C₁₋₆ alkyl, haloalkyl or halo; or a pharmaceutically acceptable salt, stereoisomer or N-oxide derivative thereof.

3. The compound of claim 2 wherein R³ is hydrogen, C₁₋₆ alkyl, C₃₋₈ cycloalkyl, —C(O)R⁵, —C(R^{a})(R^{b})OH, —SO₂R⁵, C(R^{a})(R^{b})C(O)N(R^{a})(R^{b}) or C(R^{a})(R^{b})C(O)OH, wherein said alkyl and cycloalkyl groups are optionally substituted with one to five substituents independently selected from C₁₋₆ alkyl, cyano, halo, C(O)NH₂, C(O)NHR⁴, COOH or —OR⁴; or a pharmaceutically acceptable salt, stereoisomer or N-oxide derivative thereof.

4. The compound of claim 3 wherein R³ is C₃₋₈ cycloalkyl which is optionally substituted with cyano; or a pharmaceutically acceptable salt, stereoisomer or N-oxide derivative thereof.

5. The compound of claim 4 wherein R¹ is hydrogen or C₁₋₃ alkyl; R² is hydrogen or C₁₋₃ alkyl; X is OH or hydrogen; or a pharmaceutically acceptable salt, stereoisomer or N-oxide derivative thereof.

6. The compound of claim 1 which is 1-[4′-(1-amino-2,2-difluoroethyl)biphenyl-4-yl]cyclopropanecarboxamide;
1-{4′-[(1S)-1-amino-2,2-difluoroethyl]biphenyl-4-yl}-N-cyclopropylcyclopropanecarboxamide;
1-{4′-[(1S)-1-amino-2,2,2-trifluoroethyl]-2-fluorobiphenyl-4-yl}cyclopropanecarboxamide;
1-{2-fluoro-4′-[(1R)-2,2,2-trifluoro-1-hydroxyethyl]biphenyl-4-yl}cyclopropanecarboxamide;
1-{4′-[(1S)-2,2-difluoro-1-hydroxyethyl]biphenyl-4-yl}cyclopropanecarboxamide;
1-{4′-[(1R)-2,2-difluoro-1-hydroxyethyl]biphenyl-4-yl}cyclopropanecarboxamide;
1-[4′-(1-amino-2,2-difluoroethyl)-2-fluorobiphenyl-4-yl]cyclopropanecarboxamide;
2-{4′-[(1R)-2,2,2-trifluoro-1-hydroxyethyl]biphenyl-4-yl}propanoic acid;
(2S)-2-{4′-[(1R)-2,2,2-trifluoro-1-hydroxyethyl]biphenyl-4-yl}propanoic acid;
(2S)-2-{4′-[(1R)-2,2,2-trifluoro-1-hydroxyethyl]biphenyl-4-yl}propanamide;
1-[4′-(1-amino-2,2-difluoroethyl)biphenyl-4-yl]cyclopropanecarboxylic acid;
2-[4′-(2,2-difluoro-1-hydroxyethyl)-2-fluorobiphenyl-4-yl]acetamide;
2,2-difluoro-1-[4-(4-methyl-1,3-thiazol-2-yl)phenyl]ethanol;
1-[4′-(2,2-difluoro-1-hydroxyethyl)biphenyl-4-yl]-2-methylpropan-2-ol;
1-{6-[4-(2,2-difluoro-1-hydroxyethyl)phenyl]pyridin-3-yl}cyclopropanol;
1-[4′-(2,2-difluoro-1-hydroxyethyl)-2-fluorobiphenyl-4-yl]cyclopropanol;
(1R)-1-{4′-[(1S)-1-amino-2,2,2-trifluoroethyl]biphenyl-4-yl}-2,2-difluoroethanol;
2-[4′-(2,2-difluoro-1-hydroxyethyl)-2-fluorobiphenyl-4-yl]-2-methylpropanamide;
2-[4′-(1-amino-2,2-difluoroethyl)-2-fluorobiphenyl-4-yl]-2-methylpropanamide;
2-{2-fluoro-4′-[(1R)-2,2,2-trifluoro-1-hydroxyethyl]biphenyl-4-yl}-2-methylpropanamide;
1-[4′-(2,2-difluoro-1-hydroxyethyl)biphenyl-4-yl]cyclopropanecarboxamide;
1-{4-[6-(2,2,2-trifluoro-1-hydroxyethyl)pyridin-3-yl]phenyl}cyclopropanecarboxamide;
1-{3-fluoro-4-[6-(2,2,2-trifluoro-1-hydroxyethyl)pyridin-3-yl]phenyl}cyclopropanecarboxamide;
1-biphenyl-4-yl-2,2,2-trifluoroethanol;
(1-biphenyl-4-yl-2,2,2-trifluoroethyl)amine;
2,2-difluoro-1-{4′-[(1R)-2,2,2-trifluoro-1-hydroxyethyl]biphenyl-4-yl}ethanone;
1-{4′-[(1S)-1-amino-2,2,2-trifluoroethyl]biphenyl-4-yl}-2,2-difluoroethanone;
1,1-difluoro-2-{4′-[(1R)-2,2,2-trifluoro-1-hydroxyethyl]biphenyl-4-yl}propan-2-ol;
2-{4′-[(1S)-1-amino-2,2,2-trifluoroethyl]biphenyl-4-yl}-1,1-difluoropropan-2-ol;
1-[4′-(2,2-difluoro-1-hydroxyethyl)-2-fluorobiphenyl-4-yl]cyclopropanecarbonitrile;
1-[4′-(2,2-difluoro-1-hydroxyethyl)-2-fluorobiphenyl-4-yl]cyclopropanecarboxamide 2,2-difluoro-1-[4′-(methylsulfonyl)biphenyl-4-yl]ethanol;
2,2,2-trifluoro-1-[4′-(methylsulfonyl)biphenyl-4-yl]ethane-1,1-diol;
N-cyclopropyl-1-[4′-(2,2-difluoro-1-hydroxyethyl)-2-fluorobiphenyl-4-yl]cyclopropanecarboxamide;
1-[4′-(1-amino-2,2-difluoroethyl)-2-fluorobiphenyl-4-yl]-N-cyclopropylcyclopropanecarboxamide;
1-{4′-[(1R)-1-amino-2,2,2-trifluoro-1-methylethyl]biphenyl-4-yl}cyclopropanecarboxamide;
1-[4′-(2,2,2-trifluoro-1-hydroxy-1-methylethyl)biphenyl-4-yl]cyclopropanecarboxamide;
1-{4′-[(2,4-difluorophenyl)(hydroxy)methyl]biphenyl-4-yl}cyclopropanecarboxamide;
1-{4′-[amino(2,4-difluorophenyl)methyl]biphenyl-4-yl}cyclopropanecarboxamide;
1-[4′-(2,2-difluoro-1-hydroxyethyl)-3′-fluorobiphenyl-4-yl]cyclopropanecarboxamide;
(1R)-1-[4′-(2,2-difluoro-1-hydroxyethyl)biphenyl-4-yl]-2,2,2-trifluoroethanol;
1-{4′-[(1S)-1-amino-2,2,2-trifluoroethyl]biphenyl-4-yl}-2,2-difluoroethanol;
{(1S)-2,2,2-trifluoro-1-[4′-(methylsulfonyl)biphenyl-4-yl]ethyl}amine;
1-{4′-[(1S)-1-amino-2,2,2-trifluoroethyl]biphenyl-4-yl}cyclopropanecarboxylic acid;
1-{4′-[(1S)-1-amino-2,2-difluoroethyl]biphenyl-4-yl}cyclopropanecarboxamide;
2-[4′-(1-amino-2,2,2-trifluoroethyl)biphenyl-4-yl]propanamide;
2-{4′-[(1R)-2,2,2-trifluoro-1-hydroxyethyl]biphenyl-4-yl}propanamide;
(2S)-2-{2-fluoro-4′-[(1R)-2,2,2-trifluoro-1-hydroxyethyl]biphenyl-4-yl}propanamide;
(2S)-2-{4′-[(1S)-1-amino-2,2,2-trifluoroethyl]-2-fluorobiphenyl-4-yl}propanamide;
1-biphenyl-4-yl-2,2,2-trifluoroethanol;
(1-biphen-4-yl-2,2,2-trifluoroethyl)amine;
(1R)-1-(4′-bromobiphenyl-4-yl)-2,2-difluoroethanol;
1-{4′-[(1R)-2,2-difluoro-1-hydroxylethyl]biphenyl-4-yl}-2,2,2-trifluororethanone;
1-[2-fluoro-4′-(1-hydroxy-1-methylethyl)biphenyl-4-yl]cyclopropanecarbonitrile;
1-[2-fluoro-4′-(2,2,2-trifluoro-1-hydroxyethyl)biphenyl-4-yl]cyclopropanecarbonitrile;
1-[2-fluoro-4′-(trifluoroacetyl)biphenyl-4-yl]cyclopropanecarbonitrile;
1-{2-fluoro-4′-[2,2,2-trifluoro-1-hydroxy-1-(trifluoromethyl)ethyl]biphenyl-4-yl}cyclopropanecarbonitrile;
1-[4′-(2,2,2-trifluoro-1-hydroxyethyl)biphenyl-4-yl]cyclopropanecarbonitrile;
1-[4′-(2,2,2-trifluoro-1-hydroxyethyl)biphenyl-3-yl]cyclopropanecarbonitrile;
1-(2-fluoro-4′-isopropylbiphenyl-4-yl)cyclopropanecarbonitrile;
1-[2-fluoro-4′-(2-hydroxypiperidin-2-yl)biphenyl-4-yl]cyclopropanecarbonitrile;
1-[2-fluoro-4′-(1-hydroxycyclobutyl)biphenyl-4-yl]cyclopropanecarbonitrile;
2,2,2-trifluoro-1-(4′-isopropylbiphenyl-4-yl)ethanol;
1-[2-fluoro-4′-(2-hydroxy-2-methylpropyl)biphenyl-4-yl]cyclopropanecarbonitrile;
1-[2-fluoro-3′-(2-hydroxy-2-methylpropyl)biphenyl-4-yl]cyclopropanecarbonitrile;
1-[4-(1-benzothien-3-yl)-3-fluorophenyl]cyclopropanecarbonitrile;
1-[2-fluoro-3′-(1-hydroxy-1-methylethyl)biphenyl-4-yl]cyclopropanecarbonitrile;
1-{2-fluoro-2′-[hydroxy(phenyl)methyl]biphenyl-4-yl}cyclopropanecarbonitrile;
1-{2-fluoro-4′-[hydroxy(1,3-thiazol-2-yl)methyl]biphenyl-4-yl}cyclopropanecarbonitrile;
1-[2-fluoro-3′-(3-hydroxy-3-methyl-2-oxobutyl)biphenyl-4-yl]cyclopropanecarbonitrile;
1-{3-fluoro-4-[5-(1-hydroxy-1-methylethyl)pyridin-2-yl]phenyl}cyclopropanecarbonitrile;
1-[2-fluoro-4′-(hydroxymethyl)biphenyl-4-yl]cyclopropanecarbonitrile;
1-[2-fluoro-4′-(3-hydroxy-3-methylbutyl)biphenyl-4-yl]cyclopropanecarbonitrile;
1-[4-(5-acetyl-2-thienyl)-3-fluorophenyl]cyclopropanecarbonitrile;
1-{3-fluoro-4-[5-(methylsulfonyl)pyridin-2-yl]phenyl}cyclopropanecarbonitrile;
methyl 4′-(1-cyanocyclopropyl)-2′-fluorobiphenyl-4-carboxylate;
1-(4′-benzoyl-2-fluorobiphenyl-4-yl)cyclopropanecarbonitrile;
1-(3′-acetyl-2-fluorobiphenyl-4-yl)cyclopropanecarbonitrile;
1-(3′-ethyl-2-fluorobiphenyl-4-yl)cyclopropanecarbonitrile;
1-[2-fluoro-4′-(2-hydroxyethyl)biphenyl-4-yl]cyclopropanecarbonitrile;
1-[2-fluoro-4′-(1-hydroxyethyl)biphenyl-4-yl]cyclopropanecarbonitrile;
1-[2-fluoro-3′-(2-hydroxyethyl)biphenyl-4-yl]cyclopropanecarbonitrile;
1-(2-fluoro-1,1′:3′,1″-terphenyl-4-yl)cyclopropanecarbonitrile;
1-(2-fluoro-1,1′:2′,1″-terphenyl-4-yl)cyclopropanecarbonitrile;
1-(2-fluoro-1,1′:4′,1″-terphenyl-4-yl)cyclopropanecarbonitrile;
1-(2-fluorobiphenyl-4-yl)cyclopropanecarbonitrile;
1-(2-fluoro-3′-methylbiphenyl-4-yl)cyclopropanecarbonitrile;
1-(2-fluoro-2′-methylbiphenyl-4-yl)cyclopropanecarbonitrile;
1-(4′-ethyl-2-fluorobiphenyl-4-yl)cyclopropanecarbonitrile;
1-(2-fluoro-2′-isopropylbiphenyl-4-yl)cyclopropanecarbonitrile;
1-(2-fluoro-4′-methylbiphenyl-4-yl)cyclopropanecarbonitrile;
1-[3-fluoro-4-(2-naphthyl)phenyl]cyclopropanecarbonitrile;
1-(4′-acetyl-2-fluorobiphenyl-4-yl)cyclopropanecarbonitrile;
1-[3-fluoro-4-(1H-indol-5-yl)phenyl]cyclopropanecarbonitrile;
1,1′-(2,2′-difluorobiphenyl-4,4′-diyl)dicyclopropanecarbonitrile;
1-(2-fluoro-4′-pyridin-3-ylbiphenyl-4-yl)cyclopropanecarbonitrile;
1-(2-fluoro-4′-isopropylbiphenyl-4-yl)cyclopropanecarbonitrile;
1-[4′-(1-amino-1-methylethyl)-2-fluorobiphenyl-4-yl]cyclopropanecarbonitrile;
[4′-(1-hydroxy-1-methylethyl)biphenyl-4-yl]acetonitrile;
4′-(1-hydroxy-1-methylethyl)biphenyl-4-carboxamide;
4′-(1-hydroxy-1-methylethyl)biphenyl-4-sulfonamide;
4′-(1-hydroxy-1-methylethyl)biphenyl-3-carboxamide;
2-[4-(1-benzothien-3-yl)phenyl]propan-2-ol;
1-[4′-(1-hydroxy-1-methylethyl)biphenyl-3-yl]ethanone;
2-[4-(2-naphthyl)phenyl]propan-2-ol;
1-[4′-(1-hydroxy-1-methylethyl)biphenyl-4-yl]ethanone;
2-(1,1′:4′, 1″-terphenyl-4-yl)propan-2-ol;
2-(1,1′:2′, 1″-terphenyl-4-yl)propan-2-ol;
2-(1,1′:3′, 1″-terphenyl-4-yl)propan-2-ol;
2-[4′-(methylsulfonyl)biphenyl-4-yl]propan-2-ol;
1-[4′-(1-hydroxy-1-methylethyl)biphenyl-3-yl]cyclopropanecarbonitrile;
2,2′-biphenyl-4,4′-diyldipropan-2-ol;
2-[3′-(methylsulfonyl)biphenyl-4-yl]propan-2-ol;
1-[2-fluoro-4′-(1-hydroxy-1-methylethyl)biphenyl-4-yl]cyclopropanecarboxylic acid;
2-{4′-[(methylsulfonyl)methyl]biphenyl-4-yl}propan-2-ol;
1-[4′-(1-hydroxy-1-methylethyl)biphenyl-4-yl]cyclopropanecarboxamide;
1-[2-fluoro-4′-(1-hydroxy-1-methylethyl)biphenyl-4-yl]methanesulfonamide;
1-{6-[4-(1-hydroxy-1-methylethyl)phenyl]pyridin-3-yl}cyclopropanecarbonitrile;
1-[4′-(1-hydroxy-1-methylethyl)biphenyl-3-yl]cyclopropanecarboxamide;
2-(4′-pyridin-3-ylbiphenyl-4-yl)propan-2-ol;
3-[4-(1-hydroxy-1-methylethyl)phenyl]quinoline-2-carbonitrile;
1-[4′-(1-hydroxy-1-methylethyl)biphenyl-4-yl]-N-methylcyclopropanecarboxamide;
[({1-[4′-(1-hydroxy-1-methylethyl)biphenyl-4-yl]cyclopropyl}carbonyl)(methylene)-λ⁵-azanyl]acetonitril
2-(4′-isopropoxybiphenyl-4-yl)propan-2-ol;
1-[2-fluoro-4′-(1-hydroxy-1-methylethyl)biphenyl-4-yl]cyclopropanecarboxamide;
or a pharmaceutically acceptable salt, stereoisomer or N-oxide derivative thereof.

7. A pharmaceutical composition comprising a compound of claim 1.

8. A method of treating mood disorders, depression, bipolar disorders, substance-induced mood disorders, anxiety disorders, cognitive disorders, delirium, amnestic disorders, Alzheimer's disease, schizophrenia, schizophreniform disorder, schizoaffective disorder, delusional disorder, brief psychotic disorder, shared psychotic disorder, addictive behaviors, movement disorders, akinesias, akinetic-rigid syndromes, Parkinson's disease, medication-induced parkinsonism, Gilles de la Tourette's syndrome, epilepsy, dyskinesias, chorea, myoclonus, tics, dystonia, obesity, bulimia nervosa, compulsive eating disorders, eating disorders associated with excessive food intake, osteoarthritis, repetitive motion pain, dental pain, cancer pain, myofascial pain, perioperative pain, chronic pain, neuropathic pain, post-traumatic pain, trigeminal neuralgia, migraine, attention-deficit hyperactivity disorder, conduct disorder, muscular spasms, urinary incontinence, amyotrophic lateral sclerosis, neuronal damage, ocular damage, retinopathy, macular degeneration of the eye, hearing loss, tinnitus, emesis, brain edema or sleep disorders in a mammal in need thereof by administering a therapeutically effective amount of a compound according to claim 1.

9. A pharmaceutical composition comprising a compound of claim 1 and another agent selected from the group consisting of: an anti-depressant, an anti-anxiety agent, an anti-Alzheimer's agent, a sedative, a hypnotic, an anxiolytic, an antipsychotic, a cyclopyrrolone, an imidazopyridine, a pyrazolopyrimidine, a minor tranquilizer, a melatonin agonist, a melatonin antagonist, a melatonergic agent, a benzodiazepine, a barbiturate, a 5HT-2 antagonist, levodopa, an anticholinergic, a trihexyphenidyl hydrochloride, a COMT inhibitor, an antioxidant, an A2a adenosine receptor antagonist, a cholinergic agonist, a NMDA receptor antagonist, a serotonin receptor antagonist, a monoamine oxidase inhibitor, a dopamine receptor agonist, a neuroleptic agent, an anoretic agent, a selective serotonin reuptake inhibitor, a halogenated amphetamine derivative, an opiate agonist, a lipoxygenase inhibitor, an interleukin inhibitor, an NMDA antagonist, an inhibitor of nitric oxide, a non-steroidal antiinflammatory agent, a cytokine-suppressing antiinflammatory agent, a pain reliever, a potentiator, an H2-antagonist, simethicone, aluminum hydroxide, magnesium hydroxide, a decongestant, an antitussive, and an antihistamine.

10. A method of treating mood disorders, depression, bipolar disorders, substance-induced mood disorders, anxiety disorders, cognitive disorders, delirium, amnestic disorders, Alzheimer's disease, schizophrenia, schizophreniform disorder, schizoaffective disorder, delusional disorder, brief psychotic disorder, shared psychotic disorder, addictive behaviors, movement disorders, akinesias, akinetic-rigid syndromes, Parkinson's disease, medication-induced parkinsonism, Gilles de la Tourette's syndrome, epilepsy, dyskinesias, chorea, myoclonus, tics, dystonia, obesity, bulimia nervosa, compulsive eating disorders, eating disorders associated with excessive food intake, osteoarthritis, repetitive motion pain, dental pain, cancer pain, myofascial pain, perioperative pain, chronic pain, neuropathic pain, post-traumatic pain, trigeminal neuralgia, migraine, attention-deficit hyperactivity disorder, conduct disorder, muscular spasms, urinary incontinence, amyotrophic lateral sclerosis, neuronal damage, ocular damage, retinopathy, macular degeneration of the eye, hearing loss, tinnitus, emesis, brain edema or sleep disorders in a mammal in need thereof by administering a therapeutically effective amount of a composition according to claim 9.
